(19) United States
(12) Patent Application Publication
(10) Pub. No.: US 2022/0062331 A1
(43) Pub. Date: Mar. 3, 2022

(54) TREATING IRON DEFICIENCY IN SUBJECTS AT RISK OF CARDIOVASCULAR ADVERSE EVENTS AND IRON FOR THE MANAGEMENT OF ATRIAL FIBRILLATION

(71) Applicant: PHARMACOSMOS HOLDING A/S, HOLBAEK (DK)

(72) Inventors: Claes Christian STROM, Rungsted (DK); Tobias Sidelmann CHRISTENSEN, Rockilde (DK); Thomas Goldin DINESS, Rosenvej (DK); Lars Lykke THOMSEN, Holte (DK); Darlington OKONKO, London, Cambridgeshire (GB)

(21) Appl. No.: 17/421,481

(22) PCT Filed: Feb. 10, 2020
(86) PCT No.: PCT/IB2020/051033

§ 371 Date: Jul. 8, 2021

Related U.S. Application Data
(60) Provisional application No. 62/803,455, filed on Feb. 9, 2019.

(30) Foreign Application Priority Data
Jan. 10, 2019 (GB) GB1900371.4
Jun. 12, 2019 (EP) 19179820.6

(51) Int. Cl. A61K 33/26; A61P 7/06

(52) U.S. Cl. CPC A61K 33/26; A61P 7/06

(57) ABSTRACT

The present invention relates to the field of treating iron deficiency with intravenous iron carbohydrate complexes such iron isomaltoside in subjects at risk of a cardiovascular adverse event, wherein the treatment of iron deficiency reduces the incidence of, or risk for, a cardiovascular adverse event in the subject. In another aspect, the invention provides a method for the reduction of P-wave dispersion/duration for the management of atrial fibrillation or disorders associated with atrial fibrillation (e.g., heart failure, hypertension, heart valve disease, coronary artery disease, obesity, and diabetes mellitus) in an animal suffering from such a condition which comprises administering to such an animal a therapeutically effective amount of an iron agent via the oral, intramuscular or intravenous route.

### RELATED APPLICATION DISCLOSURE

This application claims the benefit of U.S. Provisional Patent Application Ser. No. 62/803,455, filed Feb. 9, 2019, and EP Patent Application Ser. No. EP19179820.6, filed Jun. 12, 2019, each of which is hereby incorporated by reference in its entirety.

### FIELD OF THE INVENTION

The present invention relates to the field of treating iron deficiency with intravenous iron carbohydrate complexes in subjects at risk of cardiovascular adverse events. In another aspect, the invention relates to a method for preventing and treating atrial fibrillation (AF).

### BACKGROUND

Iron deficiency (ID) impairs the body's ability to produce hemoglobin, the key oxygen transporter, and impairs the function of key energy (ATP) producing enzymes. Symptoms consequently include fatigue and other signs of energy deprivation such as rapid heartbeat, shortness of breath, and chest pain.

Iron-deficiency anemia (IDA) develops when iron stores are depleted. It is widespread. About 1 billion people worldwide suffer from IDA according to the WHO. Daily oral iron is the first line therapy for most IDA patients but often fails due to lack of compliance, lack of efficacy and side effects.

High dose intravenous (IV) iron is an attractive treatment option. Patients typically require 1-3 grams of iron per year and high dose intravenous iron effectively and rapidly improves symptoms and increases hemoglobin levels. High dose intravenous iron allows treatment in one or few visits and intravenous iron is the only option for patients failing oral iron.

Iron isomaltoside 1000 (INN: ferric derisomaltose) belongs to a new generation of high dose intravenous iron products. While older low dose products (ferric gluconate and iron sucrose) required 5-20 visits, these new generation products allow for iron correction in one or two visits by fast infusion of the product.

Iron isomaltoside is a commonly used iron carbohydrate complex to treat patients with IDA who (i) have intolerance to oral iron or have had unsatisfactory response to oral iron or when there is a clinical need for rapid repletion of iron stores; or (ii) who have non-hemodialysis dependent chronic kidney disease (NDD-CKD); or to treat ID in patients with chronic kidney disease on dialysis. It is commercially available in the European Union and many other countries under the tradenames Monofer®, Monoferric® and Diafer®. A typical treatment regimen of iron isomaltoside would consist either of a single infusion of 1000 mg of iron, a dose of up to 20 mg iron/kg body weight of elemental iron given as intravenous infusion, or as an intravenous bolus injection of up to 500 mg up to three times a week, with the cumulative iron need being determined using either the Ganzoni formula or the following Table:

| Hb | Patients with bodyweight | Patients with body weight |
|---|---|---|
| (g/dL) | 50 kg to <70 kg | ≥70 kg |
| ≥10 | 1000 mg | 1500 mg |
| <10 | 1500 mg | 2000 mg |

ID has serious consequences. In chronic heart failure (CHF) patients, the risk of death or hospitalization has been reported to be increased in patients with ID relative to patients with normal iron status. Quality of life (QoL) is severely affected and improves rapidly upon restoration of iron stores. For instance, in an open-label uncontrolled design and a rather small sample size of 20 fragile elderly patients with CHF, a single fast intravenous infusion of iron isomaltoside 1000 at relatively high doses and without an initial test dose was very well tolerated and improved QoL measures. Hildebrandt et al., 2010.

The gathered experimental and clinical evidence provided the basis to consider ID as a potential therapeutic target in patients with chronic heart failure (chronic HF). Indeed, in recent years, several studies have investigated the effects of intravenous iron therapy in iron-deficient patients with chronic HF, including the FAIR-HF trial (ferric carboxymaltose assessment in patients with iron deficiency and chronic heart failure; see also Anker et al., 2009) and the CONFIRM-HF trial (ferric carboxymaltose evaluation on performance in patients with iron deficiency in combination with chronic heart failure; see also Ponikowski et al., 2015) encompassing >450 and >300 patients, respectively. Based on an aggregate data meta-analysis of five randomized controlled trials that evaluated the effects of intravenous iron therapy (using iron sucrose or ferric carboxymaltose) in iron-deficient patients with systolic HF (HFrEF), Jankowska et al., 2016 state that there is evidence that indicates that intravenous iron therapy in iron-deficient patients with systolic HF improves outcomes, alleviates HF symptoms, improves exercise capacity and quality of life, and reduces the risk of HF hospitalization. However, the number of deaths and the incidence of adverse events (AE) were similar in the five studies meta-analyzed.

There have also been attempts to explore how iron repletion confers benefits in CHF from a mechanistic point of view in particular where Hb changes are minimal. For instance, in a randomized, double-blind, placebo-controlled trial it was shown that a single total dose infusion of iron isomaltoside 1000 repleted iron stores and augmented skeletal muscle energetics at 2 weeks post-infusion. Charles-Edwards et al., 2019. Based on data from the same randomized, double-blind trial, iron repletion with a single total dose infusion of iron isomaltoside 1000 in iron-deficient CHF patients with a left ventricular ejection fraction LVEF ≤45% was observed to attenuate P wave dispersion. Jaumdally et al., 2019.

However, none of the intravenous iron trials were powered to test for an effect on major cardiovascular outcomes. The 2016 ESC Guidelines for the diagnosis and treatment of acute and chronic heart failure therefore conclude that the effect of treating iron deficiency in HFpEF/HFmrEF is unknown. Based on the findings from FAIR-HF and CONFIRM-HF, the European Society of Cardiology (ESC) guidelines recommend that intravenous ferric carboxymaltose should be considered in patients with iron deficiency (serum ferritin <100 μg/L, or ferritin between 100-299 μg/L and transferrin saturation <20%) in order to alleviate HF symptoms, and improve exercise capacity and quality of life. Ponikowski et al., 2016. Likewise, the American College of Cardiology (ACC), the American Heart Association (AHA) Task Force on Clinical Practice Guidelines and the Heart Failure Society of America (HSFA) recommend that in patients with New York Heart Association (NYHA) class II and III HF and iron deficiency (ferritin <100 ng/mL or 100 to 300 ng/mL if transferrin saturation is <20%), intravenous iron replacement might be reasonable to improve functional status and QoL. Yancy et al., 2017.

Based on this understanding, there is clearly a need for providing methods of treating iron deficiency that do not only replete iron stores but also provide benefit to patients with respect to the management of chronic heart failure.

An aspect of the invention relates to a method for preventing and treating atrial fibrillation (AF).

Atrial fibrillation is associated with considerable morbidity and mortality despite recent advances in heart treatments. It affects 33.5 million adults worldwide. At age 55, the lifetime risk for developing AF is approximately 1 in 5 with a greater incidence in older persons and patients with a predisposing disorder (heart valve disease, hypertension, heart failure, coronary artery disease, obesity, and diabetes mellitus). Due to improved detection of silent AF, an ageing population, and an increased prevalence of predisposing conditions, the global burden of AF is increasing. This has widespread health implications, as AF is a leading cause of stroke, heart failure, sudden death, hospitalisations and a poor quality of life. While better understanding of the mechanisms and subtypes of AF (paroxysmal [<7 days duration], persistent [7 days-1 year duration], long-standing [>1 year, for rhythm control] and chronic [>1 year, for rate control]) has led to newer treatments such as catheter ablation, many patients decline this invasive procedure and the outcome for those who consent is suboptimal. Thus, there is a need for newer therapies that target established features that drive AF.

Prolongation of the P-wave dispersion/duration on a surface electrocardiogram reflects an increase in the time taken for electrical impulses to be conducted in the atrial which is a hallmark feature of AF. Normally, electrical activity starts automatically at the top of the right atrium (top part of the heart) and spreads via the left atria to the atrioventricular junction where they then pass to the ventricles (bottom part of the heart). An increased P-wave dispersion reflects a delayed atrial electrical conduction and is a powerful predictor of individuals who will develop AF spontaneously, or after electrical cardioversion or cardiac surgery. This increase in atrial electrical conduction block can arise due to increased atrial scarring from the deposition of fibrotic material by inflammatory cells, but can also arise from metabolic changes in the myocardium that affect the functioning of ion channels that coordinate cardiac electrical movement. These metabolic changes are triggered by conditions that predispose to AF, but also by AF itself, and lead to a reduction in cellular energy within the atria. This atrial energetic failure is now established to be present in patients with AF, and it leads to abnormal ion channel functioning which prolongs atrial electrical conduction predisposing to AF occurrence and persistence. Therapeutic agents that can improve atrial electrical conduction, as manifested by a reduction in the P-wave dispersion/duration could prevent AF occurrence and/or induce conversion of AF to a normal sinus rhythm.

Iron supplements, also known as iron pills, injections or infusions, are used to treat and prevent iron deficiency. Because iron is found in many enzymes involved in the generation of cellular energy, iron supplements (irrespective of whether the individual is iron deficient), could enhance atrial energetics to reduce P-wave duration/dispersion and thereby prevent or treat AF.

An aspect of the invention discloses the use of iron for the reduction of P-wave dispersion/duration for the prevention and treatment of AF where the AF is either paroxysmal, persistent, long-standing, or chronic.

### SUMMARY

In one aspect of this invention, iron deficiency is treated in a subject being at risk of a cardiovascular adverse event. Accordingly, subjects are selected for treatment with iron isomaltoside not only based on the criteria commonly used to define eligibility for parenteral iron, i.e. diagnosis of ID or IDA and/or a potential lack of the ability to tolerate or absorb oral iron, but also based on their risk of experiencing a cardiovascular adverse event.

In a second aspect of this invention, the treatment of iron deficiency reduces the incidence of, or risk for, a cardiovascular adverse event in the subject. Accordingly, the subjects that are selected for treatment with iron isomaltoside based on the criteria commonly used to define eligibility for parenteral iron, i.e. diagnosis of ID or IDA and/or a potential lack of the ability to tolerate or absorb oral iron and based on their risk of experiencing a cardiovascular adverse event, benefit from a reduction of the incidence of, or risk for, a cardiovascular adverse event in the subject.

A third aspect of the invention relates to the treatment of particular groups of subjects, as defined herein, to reduce the incidence of, or risk for, a cardiovascular adverse event, as defined herein.

In line with these aspects, the present invention in particular relates to therapeutic methods of treating iron deficiency which comprise administering iron isomaltoside to selected subgroups of subjects; and combinations of iron isomaltoside 1000 with other drugs that are either used to treat subjects at risk of cardiovascular adverse events or increase the risk of such cardiovascular adverse events.

In an embodiment of said first aspect, the present invention relates to a method of treating iron deficiency in a subject being at risk of a cardiovascular adverse event, which comprises administering an effective amount of iron isomaltoside.

In a number of embodiments of said first aspect, subjects being at risk of a cardiovascular adverse event are subjects having one or more of the following risk factors:
(i) subjects having a history of myocardial infarction (MI), in particular STEMI or non-STEMI;
(ii) subjects having a history of stroke;
(iii) subjects having a history of atrial fibrillation (AF), in particular First Diagnosed AF, Proxysmal AF, or Persistent AF;
(iv) subjects having a history of congestive heart failure, in particular heart failure with reduced ejection fraction (HFrEF);
(v) subjects having a history of heart valve disorder;
(vi) subjects having a history of hypertension;
(vii) subjects having diabetes;
(viii) subjects having a history of obesity;
(ix) elderly subjects, in particular subjects of 60 years or above, 65 year or above, 70 years or above, 75 years or above, or 80 years or above;
(x) smokers;
(xi) drinkers;
(xii) subjects having hyperthyroidism and/or relatedly thyrotoxicosis;
(xiii) subjects having chronic obstructive pulmonary disorder (COPD);
(xiv) subjects having a cardiomyopathy, in particular a genetic cardiomyopathy or an acquired cardiomyopathy;
(xv) subjects having a systemic inflammation in absence of infection, wherein the systemic inflammation is in particular one that is associated with an increased C-reactive protein (CRP) above the range of about 2-3 mg/L;
(xvi) subjects on dialysis, wherein the dialysis is, in particular, hemodialysis or peritoneal dialysis;
(xvii) subjects treated with one or more of the following:
   a) modulators of the hypoxia-inducible factor (HEF) signaling pathways, including prolyl-hydroxylase inhibitors, such as daprodustat, vadadustat, roxadustat, molidustat, and desidusta;
   b) erythropoiesis-stimulating agents (ESA), such as Erythropoietin (Epo), Epoetin alfa (Procrit/Epogen), Epoetin beta (NeoRecormon), Darbepoetin alfa (Aranesp), and Methoxy polyethylene glycol-epoetin beta (Mircera); and
   c) hepcidin modulators such as a hepcidin agonist or a hepcidin antagonist;
(xviii) subjects treated with an anticoagulant and/or an NSAID;
(xix) subjects having hereditary hemorrhagic telangiectasia; or
(xx) subjects having hereditary iron refractory iron deficiency anemia.

In a particular embodiment of said first aspect, the present invention relates to a method of treating iron deficiency in a subject having a history of congestive heart failure (CHF), which comprises administering an effective amount of iron isomaltoside. According to one embodiment, the subject also has a history of myocardial infarction (MI) and/or a history of stroke. According to a preferred embodiment, CHF is heart failure with reduced ejection fraction (HFrEF).

In a further particular embodiment of said first aspect, the subject being at risk of a cardiovascular adverse event or having a history of congestive heart failure has chronic kidney disease (CKD). In another particular embodiment of said first aspect, the subject being at risk of a cardiovascular adverse event or having a history of congestive heart failure does not have chronic kidney disease (CKD). In another particular embodiment of said first aspect, the subject being at risk of a cardiovascular adverse event is a subject having chronic kidney disease (CKD) and a history of congestive heart failure (CHF).

According to one embodiment, the subject having a history of congestive heart failure has HFrEF. According to another embodiment the subject having a history of congestive heart failure has congestive heart failure in New York Heart Association (NYHA) class II-IV, in particular HFrEF-type congestive heart failure in New York Heart Association (NYHA) class II-IV. According to another embodiment the subject has congestive heart failure in NYHA class I.

In an embodiment of said second aspect, the present invention relates to a method of treating iron deficiency in a subject being at risk of a cardiovascular adverse event wherein the treatment of iron deficiency reduces the incidence of, or risk for, a cardiovascular adverse event in the subject, which method comprises administering an effective amount of iron isomaltoside.

In one embodiment of said second aspect, the cardiovascular adverse event, the incidence of, or risk for, which is reduced, is selected from the group consisting of events that affect the heart (cardiac adverse events); events that affect the peripheral vasculature (peripheral vascular adverse events); events that affect the cerebral vasculature (cerebrovascular adverse events); respiratory, thoracic and mediastinal adverse events; general adverse events; and infections and infestations. According to one embodiment, the cardiac adverse events are selected from the group consisting of congestive heart failure, atrial fibrillation, cardiac arrest, atrioventricular block, cardiac failure, sinus node dysfunction, acute myocardial infarction, bradycardia, angina pectoris, myocardial ischemia, and ventricular extrasystoles; the peripheral vascular adverse events are selected from the group consisting of hypertension, increased systolic blood pressure, increase blood pressure, increased troponin, and hypotension; the cerebrovascular adverse events are selected from the group consisting of cerebrovascular accident, cerebral infarction, and transient ischemic attack; the respiratory, thoracic and mediastinal adverse events are selected from the group consisting of dyspnea and pulmonary edema; the general adverse events are selected from the group consisting of chest pain and death; and the infections and infestations are septic shock.

In a preferred embodiment of this second aspect of the invention, the cardiovascular adverse event, the incidence of, or risk for, which is reduced, is selected from the group consisting of events that affect the heart (cardiac adverse events); events that affect the peripheral vasculature (peripheral vascular adverse events); events that affect the cerebral vasculature (cerebrovascular adverse events); and death. According to a preferred embodiment, the events that affect the heart (cardiac adverse events) are congestive heart failure, myocardial infarction, unstable angina, and arrhythmia; events that affect the peripheral vasculature (peripheral vascular adverse events) are hypertension; and events that affect the cerebral vasculature (cerebrovascular adverse events) is stroke.

In a particularly preferred embodiment of this second aspect of the invention, the cardiovascular adverse event, the incidence of, or risk for, which is reduced, is selected from the group consisting of congestive heart failure, myocardial infarction, unstable angina, arrhythmia, hypertension, hypotension, stroke, and death.

In a further particularly preferred embodiment of this second aspect of the invention, the cardiovascular adverse event, the incidence of, or risk for, which is reduced, is congestive heart failure, atrial fibrillation, hypertension, and/or cardiac arrest.

According to one embodiment, specific cardiovascular adverse events related to congestive heart failure are hospitalization or death due to congestive heart failure. In one embodiment, a specific cardiovascular adverse event related to congestive heart failure is hospitalization due to worsening congestive heart failure. The present invention is in particular directed to those CV adverse events where the CV adverse event is CHF.

In an embodiment of said third aspect, the present invention relates to a method of treating iron deficiency in a subject, wherein the treatment of iron deficiency reduces the incidence of, or risk for, a cardiovascular adverse event in the subject, which method comprises administering an effective amount of iron isomaltoside, wherein the subject is:
(A) a subject being at risk of a cardiovascular adverse event;
(B) a subject having a history of congestive heart failure (CHF); or
(C) a subject having a history of congestive heart failure (CHF) and being at risk of a cardiovascular adverse event, and
   wherein the cardiovascular adverse event, the incidence of, or risk for, which is reduced, is:
(a) selected from the group consisting of congestive heart failure, myocardial infarction, unstable angina, arrhythmia, hypertension, hypotension, stroke, and death;
(b) congestive heart failure;
(c) atrial fibrillation;
(d) hypertension; or
(e) cardiac arrest.

In a preferred embodiment of this third aspect of the invention, the subject is a subject being at risk of a cardiovascular adverse event and the cardiovascular adverse event, the incidence of, or risk for, which is reduced, is congestive heart failure.

In a further preferred embodiment of this third aspect of the invention, the subject is a subject being at risk of a cardiovascular adverse event and the cardiovascular adverse event, the incidence of, or risk for, which is reduced, is atrial fibrillation.

In a further preferred embodiment of this third aspect of the invention, the subject is a subject being at risk of a cardiovascular adverse event and the cardiovascular adverse event, the incidence of, or risk for, which is reduced, is cardiac arrest.

In a further preferred embodiment of this third aspect of the invention, the subject is a subject having a history of congestive heart failure and the cardiovascular adverse event, the incidence of, or risk for, which is reduced, is cardiac arrest or congestive heart failure or both.

In a further preferred embodiment of this third aspect of the invention, the subject is a subject having a history of congestive heart failure (CHF) and the cardiovascular adverse event, the incidence of, or risk for, which is reduced, is atrial fibrillation.

In a further preferred embodiment of this third aspect of the invention, the subject is a subject having a history of congestive heart failure (CHF) and the cardiovascular adverse event, the incidence of, or risk for, which is reduced, is cardiac arrest.

In particular embodiment of this third aspect of the invention, the subject defined herein is a subject being at risk of a cardiovascular adverse event and/or having a history of congestive heart failure, wherein the subject has chronic kidney disease (CKD).

In another particular embodiment of this third aspect of the invention, the subject defined herein the subject is a subject being at risk of a cardiovascular adverse event and/or having congestive heart failure, wherein the subject does not have chronic kidney disease (CKD).

In these embodiments, chronic kidney disease (CKD) is preferably non-dialysis dependent chronic kidney disease (NDD-CKD).

In still further preferred embodiments of this third aspect of the invention, the subject having a history of congestive heart failure has HFrEF, congestive heart failure in NYHA class II-IV, or both.

According to another embodiment the subject has congestive heart failure in NYHA class I.

In a particularly preferred embodiment of this third aspect of the invention, the cardiovascular adverse event, the incidence of, or risk for, which is reduced, is cardiovascular death and/or hospitalization due to worsening congestive heart failure.

According to the invention, iron deficiency is preferably defined as TSAT <20% and/or ferritin <100 μg/L. In one embodiment of the invention, the iron deficiency is iron-deficiency anemia.

In a further embodiment of the invention, the subject to be treated has chronic iron loss or malabsorption. In a further embodiment of said first aspect, the subject does not tolerate oral iron or for whom oral iron is not effective.

The preferred iron carbohydrate complex for use in the invention is an iron isomaltoside, especially ferric derisomaltose.

In another aspect the invention relates to a method of reducing P-wave dispersion/duration for the prevention or treatment of AF or disorders that predispose to AF (e.g., heart valve disease, hypertension, heart failure, coronary artery disease, obesity, and diabetes mellitus) in an animal suffering from such a condition which comprises administering to such an animal a therapeutically effective amount of an iron agent. The animal is preferably a mammal and most preferably a human.

The AF treated may be paroxysmal, persistent, long-standing, or chronic.

According to one embodiment, the iron agent may be administered via the oral, intramuscular, or intravenous route.

According to other embodiments the animal, preferably human, may or may not be iron deficient.

### BRIEF DESCRIPTION OF THE DRAWINGS

FIG. 1. is a flow chart of a randomized double-blind placebo-controlled trial on which an analysis was performed to evaluate the effect of intravenous iron on P-wave dispersion in patients with heart failure.

FIG. 2. is an example of a surface electrocardiogram with P-wave duration measured in each lead. P-wave dispersion is then calculated as the difference between the longest and shortest recorded P-wave duration.

FIG. 3. represents the baseline data of intravenous iron vs saline control patients.

FIG. 4. represents the results for all endpoints showing that P-wave dispersion is significantly reduced by iron.

FIG. 5. represents the results for P-wave dispersion with changes in each individual patient given. P-wave dispersion remains unchanged in the saline placebo group but is shortened in the iron group with an analysis of covariance (ANCOVA) P-value of 0.007 which is significant.

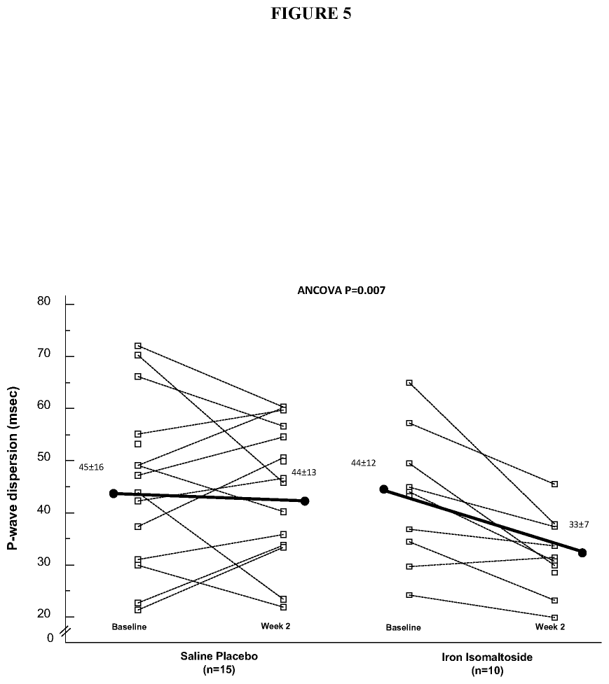

FIG. 6 is a table summarizing the analysis of the incidence of adjudicated and confirmed treatment-emergent composite cardiovascular (CV) adverse events with incidence 0.25% by preferred term for study CKD-04, study IDA-03, and the combined studies CKD-04/IDA-03.

FIG. 7 is a table summarizing the analysis of treatment-emergent congestive heart failure adverse events for study CKD-04, study IDA-04, and the combined studies CKD-04/IDA-03 in all patients; patients with or without CHF; or patients with CV risk.

FIG. 8 is a table summarizing the analysis of treatment-emergent congestive heart failure adverse events (logistic regression) for study CKD-04 and the combined studies CKD-04/IDA-03 in patients with CV risk, with or without CHF in medical history, comparing treatment with iron isomaltoside with treatment with iron sucrose.

FIG. 9 shows the percentage of patients who experienced particular treatment-emergent composite CV adverse events (iron isomaltoside 1000: left-hand bars, iron sucrose: right-hand bars).

FIG. 10 shows the percentage of patients who experienced treatment-emergent CHF adverse events (CKD-04 and IDA-03 combined; iron isomaltoside 1000: left-hand bars, iron sucrose: right-hand bars).

FIG. 11 shows a Kaplan-Meier diagram for the probability of adjudicated composite adverse cardiovascular events (CKD-04).

FIG. 12 shows the change from baseline in hemoglobin (g/dL) by patients with CHF in medical history for the combined studies CKD-04/IDA-03 (iron isomaltoside 1000: dashed line, iron sucrose: full line).

FIG. 13 shows the change from baseline in ferritin (ng/mL) by patients with CHF in medical history for the combined studies CKD-04/IDA-03 (iron isomaltoside 1000: dashed line, iron sucrose: full line).

FIG. 14 shows the change from baseline in TSAT (%) by patients with CHF in medical history for the combined studies CKD-04/IDA-03 (iron isomaltoside 1000: dashed line, iron sucrose: full line).

FIG. 15 shows the percentage of patients who experienced treatment-emergent composite CV adverse events after treatment with IIM 1000, Venofer and FCM (combined studies CKD-04/IDA-03 for IIM 1000 (Ferwon studies); studies using Venofer and FCM based on Injectafer FDA CDER report).

FIG. 16 is a table summarizing the analysis of adverse events for study CKD-04, study IDA-03, and the combined studies CKD-04/IDA-03 using IIM 1000 (Ferwon studies) compared to studies using Venofer and FCM (Injectafer FDA CDER report).

### DETAILED DESCRIPTION

In order that the present description may be more readily understood, certain terms are first defined. Additional definitions are set forth throughout the detailed description.

“Treatment” or “therapy” of a subject refers to any type of intervention or process performed on, or the administration of an active agent to, the subject with the objective of reversing, alleviating, ameliorating, inhibiting, slowing down, or preventing the onset, progression, development, severity, or recurrence of a symptom, complication, condition, or biochemical indicia associated with a disease.

A “subject” includes any human or nonhuman animal. The term “nonhuman animal” includes, but is not limited to, vertebrates such as nonhuman primates, sheep, dogs, and rodents such as mice, rats and guinea pigs. In preferred embodiments, the subject is a human. The terms, “subject” and “patient” are used interchangeably herein.

A “therapeutically effective amount” or “therapeutically effective dose” of a drug or therapeutic agent is any amount of the drug that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

For the purpose of this text, when specifying a dose in mg or g of an iron carbohydrate complex, consistent with the practice in the literature, the value refers to the amount of elemental iron provided in mg or g.

### A. Iron Carbohydrate Complexes

Described herein are therapeutic methods of treating iron deficiency which comprise administering an iron carbohydrate complex, and combinations of an iron carbohydrate complex with additional drugs, wherein the iron carbohydrate complex has certain properties and thus exerts certain effects in subjects under treatment. The methods of the invention are thus applicable to complexes that share the mechanism of action. For instance, iron carbohydrate complex should not induce significant increases in iFGF23 (intact FGF23). While in particular ferric carboxymaltose (FCM) significantly increases iFGF23 (see, for instance, WO 2013/134273 A1), results from clinical trials provide evidence that the risk for increasing iFGF23 and thus triggering iFGF23-mediated effects is low with iron isomaltoside 1000 (Monofer®). See, for instance, Wolf et al., 2019.

Accordingly, the preferred iron carbohydrate complex of this invention is iron isomaltoside (IIM). The term “iron isomaltoside” as used herein refers to colloidal complexes comprising iron, e.g., as iron oxide hydroxide, and isomaltoside in a matrix-like structure. The term “isomaltoside” as used herein refers to a hydrogenated oligoisomaltose (oligoisomaltoside).

In particular embodiments, the isomaltoside is a mixture of hydrogenated poly-/oligosaccharides having a weight average molecular weight Mw of from 500 to 7,000 Da, such as from 500 to 3,000 Da, from 700 to 1,400 Da and in particular of about 1,000 Da. The number average molecular weight (MO of such hydrogenated poly-/oligosaccharide is preferably in the range of from 400 to 1,400 Da, and 90 wt-% of these molecules have molecular weights of less than 3,500 Da, in particular less than 2,700 Da, and the molecular weights of the remaining 10% of the molecules are below 4,500 Da, in particular below 3,200 Da. For example, said hydrogenated poly-/oligosaccharide is a hydrogenated polyglucose, oligoglucose or a mixture thereof, such as a hydrogenated dextran, hydrogenated dextrin or hydrogenated oligoisomaltose (oligoisomaltoside) or a mixture thereof, with hydrogenated oligoisomaltose, particularly hydrogenated oligoisomaltose wherein the majority (such as at least 60%, e.g. from 70 to 80%) of the molecules has 3-6 monosaccharide units, being preferred. Accordingly, in preferred embodiments of the invention, the iron carbohydrate complex is an iron hydrogenated oligoisomaltose, in particular an iron(III) hydrogenated oligoisomaltose, wherein the majority (such as at least 60%, e.g. from 70 to 80%) of the oligoisomaltoside molecules has 3-6 monosaccharide units, such as iron(III) isomaltoside 1000 (INN name: ferric derisomaltose). Iron isomaltosides are typically characterized by a strong colloidal complex of iron oxide-hydroxide and hydrogenated isomaltose (isomaltoside) chains resulting in a gradual release of iron.

In the particular embodiments described above, the content of dimer saccharide of the hydrogenated poly-/oligosaccharide is preferably 2.9 wt-% or less, 2.5 wt-% or less, or 2.3 wt-% or less, in particular 2.1 wt-% or less or 1.5 wt-% or less, and most preferably 1.0 wt-% or less, based on the total weight of the hydrogenated poly-/oligosaccharide. Preferably, preparations of the hydrogenated poly-/oligosaccharide used for preparing iron carbohydrate complexes of the invention have a content of monomer saccharide of 0.5 wt-% or less. Iron hydrogenated dextran complexes prepared from such hydrogenated poly-/oligosaccharide preparations typically have an apparent molecular weight (Mₚ) in the range of from 120,000 to 180,000 Da, in particular from 130,000 to 160,000 Da. Before the hydrogenated poly-/oligosaccharide preparation is contacted with the iron preparation, the preparation can be purified by membrane processes so as to remove high molecular weight hydrogenated polysaccharides and/or low molecular weight hydrogenated oligosaccharides. In particular embodiments, the hydrogenated poly-/oligosaccharide preparation has been purified by one or more membrane processes having a cut-off value between 340 and 800 Da. In even more particular embodiments, the hydrogenated poly-/oligosaccharide preparation has been purified by one or more membrane processes using a membrane having a cut-off value that allows for holding back polysaccharides having a molecular weight above 2,700 Da, optionally followed by further hydrolysis, and followed by one or more membrane processes using a membrane having a cut-off value between 340 and 800 Da. Alternatively, the purification by said membrane processes takes place prior to the hydrogenation.

In a particularly preferred embodiment, the iron isomaltoside of the invention is a compound having the formula

{FeO_{(1-3X)}(OH)_{(1+3X)}(C₆H₅O₇³⁻)_{X}}, (C₆H₁₀O₆)_{R}(—C₆H₁₀O₅—)_{Z}(C₆H₁₃O₅)_{R}, (MeCl)_{Y} that contains H₂O, wherein

X is 0.0311±0.0062, in particular of 0.0311±0.0031;

R is 0.1400±0.0420, in particular 0.1400±0.0210;

Z is 0.4900±0.1470, in particular 0.4900±0.0735;

Y is 0.1400±0.0130, in particular 0.1400±0.0065; and

Me is a monovalent metal ion such as a sodium ion or potassium ion, and is preferably a sodium ion.

In a further particularly preferred embodiment, the iron complex compound of the invention is a compound having the formula

{FeO_{(1-3X)}(OH)_{(1+3X)}(C₆H₅O₇³⁻)_{X}}, (H₂O)_{T}, (C₆H₁₀O₆)_{R}(—C₆H₁₀O₅—)_{Z}(C₆H₁₃O₅)_{R}, (MeCl)_{Y}, wherein

X is 0.0311±0.0062, in particular 0.0311±0.0031;

T is 0.2500±0.1250, in particular 0.2500±0.24750;

R is 0.1400±0.0420, in particular 0.1400±0.0210;

Z is 0.4900±0.1470, in particular 0.4900±0.0735;

Y is 0.1400±0.0130, in particular 0.1400±0.0065; and

Me is a monovalent metal ion such as a sodium ion or potassium ion, and is preferably a sodium ion.

In particular embodiments, the iron complex has an iron content (determined for dry matter) of from 23 to 39 wt-% and is optionally present in the form of an injectable solution having about 100 mg/ml.

Iron isomaltosides are obtainable as described, for instance, in WO 2010/108493 A1 and WO 2019/048674 A1. A preferred example of iron isomaltoside is commercially available in many countries under the tradename Monofer®, Monoferric® or Diafer®.

Another particular iron carbohydrate complex for use in this invention is ferric bepectate (FBP). The term “ferric bepectate” as used herein refers to colloidal complexes comprising an iron core, e.g., as iron oxide hydroxide, coated with a hydroxyethyl-amylopectin derivative. Ferric bepectate has also been referred to as polyglucoferron. Ferric bepectate and its manufacture are disclosed, for instance, in WO 2012175608 A1. Briefly, hydroxyethyl starch is dissolved in water. Then, the pH value is adjusted to a value of 8.0 to 10.0. Afterwards, a cyanide compound is added to the hydroxyethyl starch solution. Then the solution is heated to a temperature of 80 to 99° C. and kept at this temperature for a first time period. Finally, the pH value is adjusted to a value of 2.0 to 4.0 and the solution is brought to a temperature of 50 to 90° C. and kept at this temperature for a second time period. A starch manufactured by this method is characterized in that it carries a heptonic acid residue on at least one of its termini. Thus, such starch might carry a number of heptonic acid residues per molecule, depending on the number of terminal glucosyl residues being present in the starch molecule. This heptonic acid residue increases the hydrophilicity of the hydroxyethyl starch and increases the stability of complexes formed by this hydroxyethyl starch with ligands, like for example metal ions such as iron ions. Speaking more generally, hydroxyethyl starch (HES) is a starch in which some of the hydroxyl groups of the single glucosyl residues are substituted by a hydroxyethyl residue. The modification by the heptonic acid residue takes place by converting the terminal glucosyl residue of the hydroxyethyl starch into a heptonic acid residue. Preferably, the hydroxyethyl starch used in the method has a weight average molecular weight (Mw) of less than 200,000 g/mol, in particular of less than 130,000 g/mol, in particular of less than 100,000 g/mol, in particular of less than 90,000 g/mol, in particular of less than 80 000 g/mol and very particular of less than 75,000 g/mol. A very well suited molecular weight is in the range of 55,000 g/mol to 85,000 g/mol. Such a hydroxyethyl starch has a comparatively lower molecular weight than (non-modified) hydroxyethyl starches used in the medical field at present. A suited method for determining the molecular weight of the hydroxyethyl starch is size exclusion chromatography (SEC). In a preferred embodiment, the hydroxyethyl starch has an average degree of molar substitution of 0.4 to 0.6, in particular of 0.45 to 0.55. An average degree of molar substitution of around 0.50 is particularly preferred. The average degree of molar substitution is a measure for the amount of hydroxyl groups being substituted by a hydroxyethyl residue per glucosyl residue. Since each glucose unit (or glucosyl residue) bears three hydroxyl groups, the average degree of molar substitution can be three at the maximum. An average degree of molar substitution of 0.5 indicates that (on an average or statistic basis) in each second glucosyl residue one hydroxyl group is substituted by a hydroxyethyl residue. In a preferred embodiment, the hydroxyethyl starch has a weight average molecular weight of 55,000 to 85,000 g/mol, preferably around 70,000 g/mol, and an average degree of molar substitution of 0.45 to 0.55, in particular around 0.50. Such a hydroxyethyl starch with a molecular weight of 70,000 g/mol±15,000 g/mol and an average degree of molar substitution of 0.5±0.05 can also be referred to as HES 70/0.5. A process for manufacturing this heptonic-acid modified hydroxyethyl starch, HES 70/0.5, is described in Example 1, and the formation of the iron complex in Example 2, of WO 2019/048674 A1, all of which is incorporated by reference.

Iron isomaltosides are the preferred iron carbohydrate complex for use according to the invention.

### B. Therapeutic Methods

Described herein are therapeutic methods of treating iron deficiency which comprise administering iron isomaltoside to selected subgroups of subjects and/or according to defined administration regimens. Accordingly, the present invention also relates to iron isomaltoside for use in said methods, the use of iron isomaltoside for treating iron deficiency and or the use of iron isomaltoside in the manufacture of a medicament for treating iron deficiency.

### I. Subjects to be Treated

The methods of the invention are typically performed on a subject in need thereof. A subject in need of the methods of the invention is a subject having, diagnosed with, suspected of having, or at risk for developing iron deficiency. Iron-deficiency anemia (IDA) develops when iron stores are depleted. Subjects who suffer from ID may have IDA; subjects with IDA necessarily suffer from ID. Methods to diagnose ID and IDA are well established in the art and commonly used in clinical practice.

Subjects having, diagnosed with, suspected of having, or at risk for developing iron deficiency will be given parenteral, in particular intravenous, iron in the form of an iron carbohydrate complex, i.e., iron isomaltoside according to the invention, if oral iron is not tolerated or not effective in the subject, i.e., subjects who have intolerance to oral iron or have had unsatisfactory response to oral iron. Another situation where intravenous iron is indicated is a need to deliver iron rapidly, i.e., when there is a clinical need for rapid repletion of iron stores.

### Iron Deficiency and Anemia

Iron Storage Parameters

Subjects having iron deficiency may demonstrate low or inadequate markers of systemic iron status. This means that such subjects may not have sufficient iron stored within their bodies to maintain proper iron levels. Most well-nourished, healthy people living in industrialized countries have approximately 4 to 5 grams of iron stored within their bodies. About 2.5 g of this iron is contained in hemoglobin, which carries oxygen through the blood. Most of the remaining approximately 1.5 to 2.5 grams of iron is contained in iron binding complexes that are present in all cells, but that are more highly concentrated in bone marrow and organs such as the liver and spleen. The liver's stores of iron are the primary physiologic reserve of iron in the healthy body. Of the body's total iron content, about 400 mg is utilized in proteins that use iron for cellular processes such as oxygen storage (myoglobin) or performing energy-producing redox reactions (cytochrome proteins). In addition to stored iron, a small amount of iron, typically about 3 to 4 mg, circulates through the blood plasma bound to a protein called transferrin.

Free soluble ferrous iron (iron(II) or Fe²⁺) is toxic and typically exists only in very low concentration in the body.

Individuals with iron deficiency first deplete the stored iron in the body. Because most of the iron utilized by the body is required for hemoglobin, iron-deficiency anemia is the primary clinical manifestation of iron deficiency. Oxygen transport to tissues including organs is vital and severe anemia is harmful and potentially fatal due to systemic lack of oxygen. Iron-deficient subjects will suffer, and in some instances may die, from organ damage caused by oxygen depletion well before cells run out of the iron needed for intracellular processes.

There are several markers of systemic iron status that may be measured to determine whether a subject has sufficient iron stores to maintain adequate health. These markers may be circulating iron stores, iron stored in iron-binding complexes, or both, and are also typically referred to as iron storage parameters. Iron storage parameters can include, for example, hematocrit, hemoglobin concentration (Hb), total iron-binding capacity (TIBC), transferrin saturation (TSAT), serum iron levels, liver iron levels, spleen iron levels, and serum ferritin levels. Of these, the hematocrit, hemoglobin concentration (Hb), total iron-binding capacity (TIBC), transferrin saturation (TSAT) and serum iron levels are commonly known as circulating iron stores. The liver iron levels, spleen iron levels, and serum ferritin levels are commonly referred to as stored iron or iron stored in iron-binding complexes.

It is noted that while the above blood parameters are determined in serum, they can likewise be determined in plasma. Serum and plasma levels correlate and can be converted into each other.

The present disclosure provides methods of improving one or more iron storage parameters in a subject in need thereof. The at least one iron storage parameter may be selected from serum ferritin levels, transferrin saturation (TSAT), hemoglobin concentration, hematocrit, total iron-binding capacity, iron absorption levels, serum iron levels, liver iron levels, spleen iron levels, and combinations thereof.

In one embodiment, the at least one iron storage parameter is hemoglobin concentration, and improving comprises increasing the hemoglobin concentration of the subject. In other embodiments, the at least one iron storage parameter is transferrin saturation, and improving comprises increasing the transferrin saturation of the subject. In yet other embodiments, the at least one iron storage parameter is serum ferritin levels, and improving comprises increasing the serum ferritin levels of the subject.

Serum Ferritin

The liver's stores of ferritin are the primary source of stored iron in the body. Ferritin is an intracellular protein that stores iron and releases it in a controlled fashion. Medically, the amount of ferritin present in a blood sample and/or in a sample of liver tissue reflects the amount of iron that is stored in the liver (although ferritin is ubiquitous and can be found in many other tissues within the body in addition to the liver). Ferritin serves to store iron in the liver in a nontoxic form and to transport it to areas where it is required. In a healthy subject, a normal ferritin blood serum level, sometimes referred to as the reference interval, is usually between 30-300 ng/ml for males, and 15-200 ng/ml for females. In patients at risk of cardiovascular adverse events, normal ferritin blood serum level is usually above 100 ng/mL. In an iron-deficient subject, however, serum ferritin levels are typically markedly reduced as the amount of iron available to be bound by ferritin and stored in the liver is decreased, which occurs as the body loses its ability to absorb and store iron.

The term “serum ferritin” (s-ferritin) as used herein refers to the level of ferritin in blood serum as measured using a two-site immunoenzymatic (“sandwich”) assay. Ferritin is the major iron storage protein for the body. The concentration of ferritin is directly proportional to the total iron stores of the body, resulting in serum ferritin levels becoming a common diagnostic tool in the evaluation of iron status. Subjects with iron-deficiency anemia have serum ferritin levels approximately one tenth of normal subjects, while subjects with iron overload (hemochromatosis, hemosiderosis) have serum ferritin level much higher than normal. Ferritin levels also provide a sensitive means of detecting iron deficiency at an early stage. In both adults and children, chronic inflammation results in a disproportionate increase in ferritin levels in relation to iron reserves. Elevated ferritin levels also are observed in acute and chronic liver disease, chronic renal failure and in some types of neoplastic disease.

In some embodiments, subjects treated according to the methods disclosed herein experience an increase in serum ferritin levels. In some embodiments, the present disclosure provides methods of increasing serum ferritin in a subject in need thereof, the methods comprising administering iron isomaltoside to the subject, wherein the iron isomaltoside provides an increase in serum ferritin.

In some embodiments, the iron isomaltoside provides a mean increase in serum ferritin that is greater than 100 ng/mL, greater than 110 ng/mL, greater than 120 ng/mL, greater than 130 ng/mL, greater than 140 ng/mL, greater than 150 ng/mL, greater than 160 ng/mL, greater than 170 ng/mL, greater than 180 ng/mL, greater than 190 ng/mL, or greater than 200 ng/mL at 4 or 8 weeks after treatment.

In some embodiments, the iron isomaltoside provides a mean increase in serum ferritin that is selected from less than 400 ng/mL, less than 390 ng/mL, less than 380 ng/mL, less than 370 ng/mL, less than 360 ng/mL, less than 350 ng/mL, less than 340 ng/mL, less than 330 ng/mL, less than 320 ng/mL, less than 310 ng/mL, less than 300 ng/mL, less than 290 ng/mL, less than 280 ng/mL, less than 270 ng/mL, less than 260 ng/mL, or less than 250 ng/mL at 4 or 8 weeks after treatment.

In some embodiments, the iron isomaltoside provides a mean increase in serum ferritin of 100-400 ng/mL, 100-375 ng/mL, 100-350 ng/mL, 100-325 ng/mL, 100-300 ng/mL, 100-275 ng/mL, or 150-300 ng/mL at 4 or 8 weeks after treatment.

In some embodiments, the iron isomaltoside provides a mean increase in serum ferritin that is greater than 200 ng/mL, greater than 230 ng/mL, greater than 260 ng/mL, greater than 290 ng/mL, greater than 320 ng/mL, greater than 350 ng/mL, greater than 380 ng/mL, greater than 410 ng/mL, or greater than 440 ng/mL at 1 week after treatment.

In some embodiments, the iron isomaltoside provides a mean increase in serum ferritin that is selected from less than 600 ng/mL, less than 590 ng/mL, less than 580 ng/mL, less than 570 ng/mL, less than 560 ng/mL, less than 550 ng/mL, less than 540 ng/mL, less than 530 ng/mL, less than 520 ng/mL, less than 510 ng/mL, less than 500 ng/mL, less than 490 ng/mL, less than 480 ng/mL, less than 470 ng/mL, less than 460 ng/mL, or less than 450 ng/mL at 1 week after treatment.

In some embodiments, the iron isomaltoside provides a mean increase in serum ferritin of 200-600 ng/mL, 250-600 ng/mL, 300-600 ng/mL, 350-600 ng/mL, or 400-600 ng/mL at 1 week after treatment.

Transferrin Saturation (TSAT)

In addition to stored iron, a small amount of iron, typically about 3 to 4 mg, circulates through the blood plasma bound to a protein called transferrin. Therefore, serum iron (s-iron) levels can be represented by the amount of iron circulating in the blood that is bound to the protein transferrin. Transferrin is a glycoprotein produced by the liver that can bind one or two ferric iron (iron(III) or Fe3+) ions. It is the most prevalent and dynamic carrier of iron in the blood, and therefore is an essential component of the body's ability to transport stored iron for use throughout the body. Transferrin saturation (or TSAT) is measured as a percentage and is calculated as the ratio of serum iron and total iron-binding capacity, multiplied by 100. This value tells a clinician how much serum iron is actually bound to the total amount of transferrin that is available to bind iron. For instance, a TSAT value of 35% means that 35% of the available iron-binding sites of transferrin in a blood sample are occupied by iron. In a healthy subject, typical TSAT values are approximately 15-50% for males and 12-45% for females. In patients at risk of cardiovascular adverse events, normal TSAT values are typically above 20%. In an iron-deficient subject, however, TSAT values are typically markedly reduced as the amount of iron available to be bound by transferrin is decreased, which occurs as the body loses its ability to absorb and store iron. In some embodiments, the TSAT value is below 20% and/or the ferritin concentration is <100 μg/L.

In some embodiments, subjects treated according to the methods disclosed herein experience an increase in TSAT values. In some embodiments, the present disclosure provides methods of increasing TSAT in a subject in need thereof, the methods comprising administering iron isomaltoside to the subject, wherein the iron isomaltoside provides an increase in TSAT in the subject.

In some embodiments, the iron isomaltoside provides a mean increase in TSAT that is greater than 1%, greater than 1.5%, greater than 2%, or greater than 2.5% at 4 or 8 weeks after treatment.

In some embodiments, the iron isomaltoside provides a mean increase in TSAT that is less than 5%, less than 4%, or less than 3% at 4 or 8 weeks after treatment.

In some embodiments, the iron isomaltoside provides a mean increase in TSAT of 1-5%, 1.5-4%, or 2-3% at 4 or 8 weeks after treatment.

In some embodiments, the iron isomaltoside provides a mean increase in TSAT that is greater than 5%, greater than 6%, or greater than 7% at 1 week after treatment.

In some embodiments, the iron isomaltoside provides a mean increase in TSAT that is less than 20%, less than 19%, less than 18%, less than 17%, less than 16%, or les than 15% at 1 week after treatment.

In some embodiments, the iron isomaltoside provides a mean increase in TSAT of 5-20%, or 5-15% at 1 week after treatment.

Hematocrit

The hematocrit, also referred to as packed cell volume or erythrocyte volume fraction, is the volume percentage of red blood cells in the blood. For healthy subjects, the hematocrit is typically about 45% of blood volume for men and about 40% of blood volume for women. In iron-deficient subjects, however, the hematocrit is often significantly depleted due to poor iron absorption and/or poor iron storage capacity.

The iron isomaltoside disclosed herein may be administered to subjects to increase hematocrit. The exact timing of administration will necessarily vary from subject to subject, depending upon, for example, the severity of the iron deficiency experienced by the subject, the level of iron absorption the subject is or is not experiencing, and the judgment of the treating health care professional. In some embodiments, the present disclosure provides methods of increasing hematocrit in a subject in need thereof, the methods comprising administering iron isomaltoside to the subject, wherein the iron isomaltoside provides for an increase in the hematocrit of the subject. In some embodiments, the increase is from 1% to 30%, from 1% to 15%, from 1% to 12%, from 1% to 10%, from 1% to 9%, from 1% to 8%, from 1% to 7%, from 1% to 6%, from 1% to 5%, from 1% to 4%, from 1% to 3%, or from 1% to 2%.

Hemoglobin Concentration

Hemoglobin concentration, also referred to as the mean corpuscular hemoglobin concentration or MCHC, is a measure of the concentration of hemoglobin protein in a given volume of packed red blood cells. It is typically calculated by dividing the total amount of hemoglobin protein by the hematocrit. Hemoglobin concentration may also be measured as a mass or weight fraction and presented as a percentage (%). Numerically, however, the mass or molar measure of hemoglobin concentration and the mass or weight fraction (%) are identical, assuming a red blood cell density of 1 g/ml and negligible hemoglobin loss in the blood plasma. For healthy subjects, a typical mass or molar measure of hemoglobin concentration ranges from 32 g/dl-36 g/dl, or from 4.9 mmol/L to 5.5 mmol/L, respectively. In an iron-deficient subject, however, the hemoglobin concentration can be greatly reduced as the body loses its ability to absorb and store iron.

In some embodiments, subjects treated according to the methods disclosed herein experience an increase in hemoglobin concentration. In some embodiments, the present disclosure provides methods of increasing hemoglobin concentration in a subject in need thereof, the methods comprising administering iron isomaltoside to the subjects, wherein the iron isomaltoside provides an increase in hemoglobin concentration in the subjects.

In some embodiments, the iron isomaltoside provides a mean increase in hemoglobin concentration of 0.1-5.0 g/dL, 0.1-4.0 g/dL, 0.1-3.0 g/dL, or 0.1-2.0 g/dL at 1 week after treatment.

In some embodiments, the iron isomaltoside provides a mean increase in hemoglobin concentration greater than 0.1 g/dL, greater than 0.2 g/dL, greater than 0.3 g/dL, greater than 0.4 g/dL, greater than 0.5 g/dL, greater than 0.6 g/dL, greater than 0.7 g/dL, greater than 0.8 g/dL, or greater than 0.9 g/dL at 1 week after treatment.

In some embodiments, the iron isomaltoside provides a mean increase in hemoglobin concentration of less than 1.0 g/dL, less than 0.9 g/dL, less than 0.8 g/dL. In some embodiments, less than 0.7 g/dL, less than 0.6 g/dL, less than 0.5 g/dL, less than 0.4 g/dL, or less than 0.3 g/dL at 1 week after treatment.

In some embodiments, the iron isomaltoside provides a mean increase in hemoglobin concentration of 0.5-5.0 g/dL, 0.5-4.0 g/dL, 0.5-3.0 g/dL, or 0.5-2.0 g/dL at 4 or 8 weeks after treatment.

In some embodiments, the iron isomaltoside provides a mean increase in hemoglobin concentration greater than 0.5 g/dL, greater than 0.7 g/dL, greater than 0.9 g/dL, greater than 1.1 g/dL, greater than 1.3 g/dL, or greater than 1.5 g/dL at 4 or 8 weeks after treatment.

In some embodiments, the iron isomaltoside provides a mean increase in hemoglobin concentration of less than 2.0 g/dL, less than 1.9 g/dL, less than 1.8 g/dL. In some embodiments, less than 1.7 g/dL, less than 1.6 g/dL, less than 1.5 g/dL, less than 1.4 g/dL, or less than 1.3 g/dL at 4 or 8 weeks after treatment.

Total Iron Binding Capacity (TIBC)

Total iron-binding capacity (TIBC) is a measure of the blood's capacity to bind iron with the protein transferrin. TIBC is typically measured by drawing a blood sample and measuring the maximum amount of iron that the sample can carry. Thus, TIBC indirectly measures transferrin, which is a protein that transports iron in the blood. For healthy subjects, a typical mass or molar measure of TIBC is in the range of 250-370 μg/dL or 45-66 μmon, respectively. In iron-deficient subjects, however, the TIBC is typically increased above these levels, as the body must produce more transferrin in an attempt to deliver iron to erythrocyte precursor cells to produce hemoglobin.

In some embodiments, subjects treated according to the methods disclosed herein experience a reduction in TIBC. In some embodiments, the present disclosure provides methods of reducing TIBC in subjects in need thereof, the methods comprising administering iron isomaltoside to the subject, wherein the iron isomaltoside provides for a reduction in the TIBC of the subject.

In some embodiments, the reduction is from 0.1% to 30%, from 0.1% to 28%, from 0.1% to 26%, from 0.1% to 25%, from 0.1% to 24%, from 0.1% to 23%, from 0.1% to 22%, from 0.1% to 21%, from 0.1% to 20%, from 0.1% to 15%, from 0.1% to 10%, or from 0.1% to 5%.

Subjects at risk of cardiovascular adverse events will generally be subject to or at risk of systemic inflammation, which can complicate the assessment of iron parameters. As disclosed herein, in subjects at risk of cardiovascular adverse events, normal iron parameters would generally be considered to be a TSAT >20% and/or a serum ferritin >100 μg/L. According to one embodiment of this invention, treatment is appropriate if TSAT is less than 20% and/or serum ferritin is less than 100 μg/L or in a related embodiment if TSAT is less than 20% and serum ferritin is less than 300 μg/L. In a preferred embodiment, TSAT is less than 20% and/or serum ferritin is less than 100 μg/L. Treatment criteria may also include an upper limit on serum ferritin beyond which re-dosing is not recommended such as serum ferritin of no more than 300 μg/L, of no more than 400 μg/L, of no more than 500 μg/L, or of no more than 600 μg/L. Re-treatment criteria may also include higher limits for ferritin and TSAT than the criteria for treatment. For example, in one preferred embodiment, subsequent to the administration of the initial effective dose, subjects will receive a further dose if TSAT is less than 25% and/or serum ferritin is less than 100 μg/L and provided that serum ferritin is not higher than 400 μg/L.

Symptoms

Symptoms of iron deficiency can occur before the condition has progressed to iron-deficiency anemia. Symptoms of iron deficiency can include, for example, fatigue, dizziness, pallor, hair loss, irritability, weakness, pica, brittle or grooved nails, Plummer-Vinson syndrome (painful atrophy of the mucous membrane covering the tongue, pharynx and esophagus), impaired immune function, pagophagia, and restless legs syndrome, among others.

Subjects treated according to the methods disclosed herein will experience an improvement in iron deficiency. In some embodiments, subjects treated according to the methods disclosed herein experience a decrease in iron deficiency. This decrease may occur as the total amount of iron in the body of the subject is increased through the administration of the iron isomaltoside disclosed herein. In some embodiments, subjects treated according to the methods disclosed herein experience a decrease in, or an elimination of, one or more symptoms of iron deficiency, wherein the symptoms are selected from fatigue, dizziness, pallor, hair loss, irritability, weakness, pica, brittle or grooved nails, Plummer-Vinson syndrome (painful atrophy of the mucous membrane covering the tongue, pharynx and esophagus), impaired immune function, pagophagia, restless legs syndrome and combinations of the foregoing.

In some embodiments, the iron deficiency is iron-deficiency anemia. Iron-deficiency anemia is characterized by low levels of circulating red blood cells and can be caused by insufficient dietary intake, absorption and/or storage of iron. Red blood cells, which contain iron bound in hemoglobin proteins, and are typically not formed when the amount of iron in the body is deficient.

Iron-deficiency anemia is typically characterized by pallor (pale color resulting from reduced oxyhemoglobin in the skin and mucous membranes), fatigue, lightheadedness, and weakness. However, signs of iron-deficiency anemia can vary between subjects. Because iron deficiency tends to develop slowly, adaptation to the disease can occur and it can go unrecognized for some time. In some instances, subjects can develop dyspnea (trouble breathing), pica (unusual obsessive food cravings), anxiety often resulting in OCD-type compulsions and obsessions, irritability or sadness, angina, constipation, sleepiness, tinnitus, mouth ulcers, palpitations, hair loss, fainting or feeling faint, depression, breathlessness on exertion, twitching muscles, pale yellow skin, tingling (numbness) or burning sensations, missed menstrual cycle(s), heavy menstrual period(s), slow social development, glossitis (inflammation or infection of the tongue), angular cheilitis (inflammatory lesions at the mouth's corners), koilonychia (spoon-shaped nails) or nails that are weak or brittle, poor appetite, pruritus (generalized itchiness), Plummer-Vinson syndrome (painful atrophy of the mucous membrane covering the tongue, pharynx and esophagus), and restless legs syndrome, among others.

Anemia is typically diagnosed based on a complete blood count measured from a blood sample from a subject. Typically, automatic counters are utilized that report the total number of red blood cells in a sample, the hemoglobin level, and the size of the red blood cells by flow cytometry. However, a stained blood smear on a microscope slide can be examined using a microscope in order to count the total number of red blood cells in a sample and diagnose anemia. In many countries, four parameters (red blood cell count, hemoglobin concentration, mean corpuscular volume and red blood cell distribution width) are measured to determine the presence of anemia. The World Health Organization has set certain threshold values for hemoglobin levels (Hb), such that when a subject's hemoglobin levels fall below those values, a diagnosis of anemia may be made. Those values are: for children 0.5-5.0 yrs of age, Hb=11.0 g/dL or 6.8 mmol/L; for children 5-12 yrs years of age, Hb=11.5 g/dL or 7.1 mmol/L; for teens 12-15 yrs of age, Hb=12.0 g/dL or 7.4 mmol/L; for non-pregnant women 15 years of age and older, Hb=12.0 g/dL or 7.4 mmol/L; for pregnant women, Hb=11.0 g/dL or 6.8 mmol/L; and for men greater than 15 yrs of age, Hb=13.0 g/dL or 8.1 mmol/L.

Subjects treated according to the methods disclosed herein may experience an improvement in anemia. Subjects treated according to the methods disclosed herein may experience an improvement in iron-deficiency anemia. In some embodiments, subjects treated according to the methods disclosed herein experience a decrease in one or more symptoms of anemia or iron-deficiency anemia. In some embodiments, subjects treated according to the methods disclosed herein experience the elimination of one or more symptoms of anemia or iron-deficiency anemia. In some embodiments, the one or more symptoms of anemia or iron-deficiency anemia are selected from pallor, fatigue, lightheadedness, weakness, dyspnea, pica, anxiety, irritability or sadness, angina, constipation, sleepiness, tinnitus, mouth ulcers, palpitations, hair loss, fainting or feeling faint, depression, breathlessness on exertion, twitching muscles, pale yellow skin, tingling (numbness) or burning sensations, missed menstrual cycle(s), heavy menstrual period(s), slow social development, glossitis, angular cheilitis, koilonychia, poor appetite, pruritus, Plummer-Vinson syndrome, restless legs syndrome and combinations of the foregoing.

In some embodiments, subjects treated according to the methods disclosed herein may experience an improvement in anemia and/or iron-deficiency anemia because hemoglobin levels are raised and/or maintained above a threshold level. In some embodiments, a method of treating anemia is disclosed, the method comprising administering iron isomaltoside to the subject, wherein the iron isomaltoside provides a hemoglobin level in the subject that is at or above a level ranging from 11.0 g/dL-13.0 g/dL, including a level selected from 11.0 g/dL, 11.5 g/dL, 12.0 g/dL, and 13.0 g/dL. In some embodiments, a method of treating anemia is disclosed, the method comprising administering iron isomaltoside to the subject, wherein the iron isomaltoside provides a hemoglobin level in the subject that is at or above a level selected from 6.8 mmol/L, 7.1 mmol/L, 7.4 mmol/L, and 8.1 mmol/L. In some embodiments, a method of treating anemia in a male subject is disclosed, the method comprising administering iron isomaltoside to the male subject, wherein the iron isomaltoside provides a hemoglobin level in the male subject that is at or above a level selected from 13.0 g/dL and 8.1 mmol/L. In some embodiments, a method of treating anemia in a female subject is disclosed, the method comprising administering iron isomaltoside to the female subject, wherein the iron isomaltoside provides a hemoglobin level in the female subject that is at or above a level selected from 12.0 g/dL and 7.4 mmol/L.

### Risk Factors for a Cardiovascular Adverse Event

A particular group of subjects having iron deficiency as disclosed herein that are amenable to treatment according to the present invention is characterized as being at risk of a cardiovascular adverse event.

According to the invention, a subject being at risk of a cardiovascular adverse event is a subject having a risk for one or more events selected from the group consisting of events that affect the heart (cardiac adverse events), such as congestive heart failure (CHF), in particular CHF requiring hospitalization or medical intervention, myocardial infarction, unstable angina, in particular angina requiring hospitalization, or arrhythmia; events that affect the peripheral vasculature (peripheral vascular adverse events), such as hypertension and hypotension; events that affect the cerebral vasculature (cerebrovascular adverse events), such as stroke; and/or general adverse events, such as death.

More specifically, a subject being at risk of a cardiovascular adverse event according to the invention is a subject having one or more of the following risk factors for cardiovascular events and in particular the cardiovascular events defined herein:

Chronic Kidney Disease (CKD)

Subjects with a glomerular filtration rate (GFR)<60 ml/min/1.73 m² for 3 months are classified as having CKD, irrespective of the presence or absence of kidney damage. Those subjects with CKD who require either dialysis or kidney transplantation are typically referred to as end-stage renal disease (ESRD) subjects. Therefore, a subject is traditionally classified as an ESRD subject when he or she reaches the conclusion of the non-dialysis dependent, earlier stages, of CKD. Prior to then, those subjects are referred to as non-dialysis dependent CKD subjects. Non-dialysis-dependent CKD (NDD-CKD) subjects are those who have been diagnosed with an early stage of chronic kidney disease and who have not yet been medically directed to undergo dialysis. The U.S. National Kidney Foundation has defined 5 stages of chronic kidney disease. Typically, subjects having CKD progress through stages 1 through 4 before dialysis is medically necessary. However, subjects with an advanced stage of CKD, such as stage 5, who have not yet started dialysis or who have not been recommended for transplantation are also typically referred to as non-dialysis dependent CKD subjects.

As used herein, NDD-CKD is intended to cover all subjects who have been diagnosed with chronic kidney disease but who are not undergoing dialysis during the administration of iron isomaltoside. Such subjects can include, for example, subjects who have never been subjected to dialysis and, in some embodiments, subjects who have been subjected to dialysis but who are not undergoing dialysis during the administration of iron isomaltoside.

Cardiovascular disease is a frequent cause of death in subjects with chronic kidney disease (CKD). Despite the high prevalence of traditional risk factors for atherosclerosis in subjects with CKD, heart failure, arrhythmia, and sudden cardiac death constitute a disproportionately greater burden of cardiovascular related mortality in subjects with CKD compared to coronary artery disease (CAD). Left ventricular hypertrophy (LVH) and left ventricular dysfunction can be a consequence of having atherosclerosis but are also common non-atherosclerotic mechanisms of cardiovascular injury in CKD. Cardiac disease, including coronary artery disease, left ventricular hypertrophy (LVH) and heart failure (HF), is common in subjects with chronic kidney disease (CKD). LVH appears to be increasingly prevalent as the glomerular filtration rate (GFR) declines and with increased dialysis usage.

LVH is an important predictor of mortality in subjects with CKD and anemia has emerged as an important, independent risk factor for the development and progression of LVH and HF in CKD, and of adverse cardiovascular outcomes, including mortality.

The presence of LVH is important clinically because it is associated with increases in the incidence of heart failure, ventricular arrhythmias, death following myocardial infarction, decreased LV ejection fraction, sudden cardiac death, aortic root dilation, and a cerebrovascular event. In general, the development of heart failure with LVH results from depressed left ventricular systolic function and/or diastolic dysfunction. The deleterious effect of left ventricular remodeling may be an important determinant of progression to overt heart failure.

Without intending to be limited by theory, potential mechanisms that may explain the relationship between anemia and the development of LVH include effects of reduced oxygen delivery to the myocardium, perhaps leading to increased myocyte necrosis and apoptosis, anemia-related increased cardiac output and reduced systemic vascular resistance, increased oxidative stress, less effective oxidative phosphorylation, and activation of the sympathetic nervous system.

In CKD, increasingly severe anemia is associated with more frequent and severe LVH, LV dilatation, HF, and poorer all-cause and cardiac prognosis in subjects with CKD and cardiac disease. Given that increasing anemia in CKD is associated with increasingly severe degrees of LVH and heart failure, in accordance with aspects of the present disclosure, correcting deficiencies related to anemia have a beneficial impact on the clinical features of HF and LVH. This includes the clinical manifestations of HF and the subsequent development and progression of LVH.

Several, mostly uncontrolled short-term studies, have suggested that improving anemia to Hb levels of about 10 to 12 g/dL with erythropoietin in subjects with HF and CKD improves clinical manifestations of HF and reduces hospitalization rates but fails to improve hard endpoints in CHF subjects. However, given the rather unexpected adverse outcomes associated with the use of erythropoietin, there is the possibility of an increased subject risk of morbidity and/or mortality with erythropoietin treatment aimed at meeting normal or near-normal Hb levels.

LVH, which is itself a powerful prognostic marker for adverse cardiovascular outcomes in subjects with CKD, appears to regress with improvement in Hb levels from less than 10 g/dL to levels up to above 10 g/dL in some subjects. Again, without intending to be limited, it does not appear that raising the Hb further to more normal levels leads to further regression of LVH or clinical improvement. Baseline geometry of LVH may be an important factor in determining the subsequent clinical response to anemia correction in subjects with LVH.

Congestive Heart Failure (CHF)

“Congestive heart failure” and “cardiac failure congestive”, a standardized MedDRA term, are used interchangeably herein.

Heart failure is a clinical syndrome characterized by typical symptoms (e.g. breathlessness, ankle swelling and fatigue) that may be accompanied by signs (e.g. elevated jugular venous pressure, pulmonary crackles and peripheral oedema) caused by a structural and/or functional cardiac abnormality, resulting in a reduced cardiac output and/or elevated intracardiac pressures at rest or during stress. Thus, the current definition of HF restricts itself to stages at which clinical symptoms are apparent. Before clinical symptoms become apparent, subjects can present with asymptomatic structural or functional cardiac abnormalities, such as systolic or diastolic left ventricular (LV) dysfunction, which are precursors of HF. Recognition of these precursors is important because they are related to poor outcomes, and starting treatment at the precursor stage may reduce mortality in subjects with asymptomatic systolic LV dysfunction.

“Congestive HF” is a term that is used to describe chronic heart failure, in particular if there is evidence of volume overload, most often displayed as peripheral or pulmonary edema. Congestive heart failure (CHF) may be heart failure with reduced ejection fraction, LVEF <40% (HFrEF), heart failure with preserved ejection fraction, LVEF ≥50% (HFpEF), or heart failure with mid-range ejection fraction, LVEF 40-49% (HFmrEF). In a particular embodiment of the invention, the congestive heart failure (CHF) is heart failure with reduced ejection fraction, LVEF <40% (HFrEF).

Congestive heart failure (CHF) may also be classified in accordance with the New York Heart Association (NYHA) Classification which classifies patients in one of four categories based on how much they are limited during physical activity. Patients having no symptoms and no limitation such as undue fatigue, palpitation, or dyspnea (shortness of breath), in ordinary physical activity, e.g. walking, climbing stairs etc., are assigned to NYHA class I. Patients with mild symptoms (mild shortness of breath and/or angina) and slight limitation of physical activity are assigned to NYHA class II. Patients having a marked limitation in activity due to symptoms, even during less-than-ordinary activity, e.g. walking short distances of 20 to 100 m, and which are comfortable only at rest are assigned to NYHA class III. Patient who experience symptoms even while being at rest and are unable to carry on any physical activity without discomfort are assigned to NYHA class W. In a particular embodiment of the invention, the congestive heart failure (CHF) is congestive heart failure in NYHA class II-IV. According to another embodiment the subject has congestive heart failure in NYHA class I.

Atrial Fibrillation (AF)

Atrial fibrillation (AF) can be First Diagnosed AF (AF that has not been diagnosed before, irrespective of the duration of the arrhythmia or the presence and severity of AF-related symptoms), Paroxysmal AF (Self-terminating, in most cases within 48 hours. Some AF paroxysms may continue for up to 7 days. AF episodes that are cardioverted within 7 days should be considered paroxysmal), and Persistent AF (AF that lasts longer than 7 days, including episodes that are terminated by cardioversion, either with drugs or by direct current cardioversion, after 7 days or more), Long-standing persistent AF (Continuous AF lasting for ≥1 year when it is decided to adopt a rhythm control strategy), or Permanent AF (AF that is accepted by the patient (and physician). According to a particular embodiment of the invention, AF is First Diagnosed AF, Paroxysmal AF or Persistent AF.

AF is associated with an increased risk of death, cardiovascular adverse events and renal diseases, including a higher risk of ischaemic heart disease, chronic kidney disease, sudden cardiac death, stroke, or incident congestive heart failure.

Hypertension

Hypertension is defined as an increase in systolic blood pressure >20 mm Hg that results in a value >180 mm Hg or an increase in diastolic blood pressure >15 mm Hg that results in a value >105 mm Hg.

Hypertension is one of the leading risk factors for heart diseases, in particular, a risk factor for stroke. It causes about 50% of ischemic strokes and increases the risk of hemorrhagic stroke.

There are several mechanisms associated with hypertension that make it such a high risk factor for a cardiovascular adverse event. For example, hypertension stresses blood vessels due to narrowing or clogging them. This can lead to atherosclerosis or the creation of weak points in the vessel that rupture easily or balloon out the artery wall resulting in an aneurism.

Myocardial Infarction (MI)

Myocardial infarction (MI) is defined as myocardial cell death due to prolonged ischemia. After the onset of myocardial ischemia histological cell death does not occur immediately, but takes a finite period of time to develop. After several hours complete necrosis of myocardial cells can be recognized. MI may be the first manifestation of coronary artery disease (CAD).

MI is a term that is used to describe the presence of characteristic changes in cardiac enzyme markers in the setting of either temporally related symptoms of an acute coronary syndrome or electrocardiographic (ECG) changes consistent with either ischemia or infarction. Cardiac Troponin cTn (I or T) which has high myocardial tissue specificity and high clinical sensitivity is used as a preferred biomarker. Alternatively, if a cTn assay is not available, the MB fraction of creatine kinase (CKMB), measured by mass assay, can be used. In both assays, an increase in cTn or CKMB concentration is defined as a value exceeding the 99^{th} percentile of a normal reference population (upper reference limit (URL)). This discriminatory 99^{th} percentile is designated as the decision level for the diagnosis of MI.

MI in patients with chest discomfort, or other ischaemic symptoms that develop ST elevation in two contiguous leads, is designated as an ‘ST elevation MI’ (STEMI). Patients without ST elevation at presentation are usually designated as having a ‘non-ST elevation MI (non-STEMI). In a preferred embodiment, the myocardial infarction (MI) is ST-segment elevation myocardial infarction (STEMI) or non-STEMI.

Subjects having a history in MI are one of the highest risk groups for further cardiovascular adverse event. In particular, subjects having survived MI, i.e. having a history of MI, are at increased risk of recurrent infarctions and have an annual death rate which is six times higher than that in people of the same age who do not have coronary heart disease.

Stroke

Stroke is classically characterized as a neurological deficit attributed to an acute focal injury of the central nervous system by a vascular cause, including cerebral infarction, intracerebral hemorrhage, and subarachnoid hemorrhage.

Subjects having a history in stroke, i.e. subjects who have suffered a stroke, remain at a high risk for further cardiovascular adverse event, in particular for a further stroke.

Heart Valve Disorders

Aortic stenosis is the most common primary valve disease leading to surgery or catheter intervention in Europe and North America, with a growing prevalence due to the ageing population.

Mitral regurgitation (MR) is the second-most frequent indication for valve surgery in Europe. It is possible to distinguish primary from secondary MR, particularly regarding surgical and transcatheter interventional management. In primary MR, one or several components of the mitral valve apparatus are directly affected. The most frequent etiology is degenerative (prolapse, flail leaflet). Endocarditis as one of the causes of primary MR. Secondary MR (also known as functional MR) is defined as MR due to primary left ventricular (LV) dysfunction with normal mitral valve leaflets and chords. LV dysfunction may be due to coronary heart disease (CHD) or (non-ischemic) cardiomyopathy.

Subjects having a history of heart valve defects, e.g., from birth, have an increased likelihood for developing valve problems. Heart valve disease can cause many complications, including cardiovascular adverse events such as HF, stroke, blood clots, arrhythmia, or death.

Diabetes

Marked hyperglycemia is associated with symptoms including frequent urination, thirst, blurred vision, fatigue and recurring infections. Beyond alleviating symptoms, the aim of blood glucose lowering is to reduce long-term complications of diabetes. Diabetes mellitus type 2 (also known as type 2 diabetes) is a long-term metabolic disorder that is characterized by high blood sugar, insulin resistance, and relative lack of insulin. Common symptoms include increased thirst, frequent urination, and unexplained weight loss. Type 1 diabetes, once known as juvenile diabetes or insulin-dependent diabetes, is a chronic condition in which the pancreas produces little or no insulin. Different factors, including genetics and some viruses, may contribute to type 1 diabetes. Although type 1 diabetes usually appears during childhood or adolescence, it can develop in adults. Despite active research, type 1 diabetes has no cure. Treatment focuses on managing blood sugar levels with insulin, diet and lifestyle to prevent complications.

Having diabetes increases the risk for a cardiovascular adverse event two to four times compared to people without diabetes. Cardiovascular adverse event are the leading cause of mortality for people with diabetes. This is in particular due to hypertension, abnormal blood lipids and obesity, all risk factors for cardiovascular adverse events, which is widely spread in subjects suffering from diabetes.

For example, uncontrolled diabetes can cause damages to the blood vessels which can lead to atherosclerosis and hypertension. High glucose levels increase the likelihood for the buildup of fatty deposits (atheroma) which can lead to coronary heart disease and heart attack if they occur in the coronary arteries. Subjects with diabetes are more likely to have a heart attack or stroke, than subjects without diabetes and the risk of heart failure is increased compared to subjects without diabetes. Diabetes inhibits the protective effects of estrogen which can increase the risk of a cardiovascular adverse event in premenopausal women who have diabetes.

Obesity

“Obesity” is a condition characterized by excessive body weight to the extent when the body mass index (BMI), a measurement obtained by dividing a person's weight by the square of the person's height, is over 30 kg/m2. Having a BMI of greater than 30 kg/m², i.e. being obese, there is serious risk for developing hypertension (e.g., due to intra-abdominal fat), diabetes and atherosclerosis which are risk factors for a cardiovascular adverse event. The term “history of obesity” refers to a patient who is determined to be presently obese according to the aforementioned criteria, and/or has previously been determined to be obese.

For example, being overweight, a high intake of salt increases the risk of a cardiovascular adverse event, in particular, as dietary salt is a significant factor in raising blood pressure which can lead to hypertension and the associated risks.

Age, Smoking and Drinking

Subjects, who are elderly, a smoker, and/or a drinker, have an increased risk for a cardiovascular adverse event.

According to a particular embodiment of the invention, the elderly subject is 60 years or above, 65 year or above, 70 years or above, 75 years or above, or 80 years or above. The risk of stroke doubles every decade after age 55. The systolic blood pressure is an important predictor of the risk of a cardiovascular event by getting older.

A drinker refers to a subject with elevated alcohol intact, in particular a subject having more than 7 drinks per week.

Drinkers consuming too much alcohol can suffer from, or develop, problems such as increased blood pressure, acute myocardial infarction or cardiomyopathy. Further, abuse of alcohol has been shown to damage heart muscle and increase the risk of stroke and cardiac arrhythmia.

A smoker is a person who used to be a regular smoker of tobacco and/or who is currently a regular smoker of tobacco.

Since the 1940s it is known that smoking is linked to cardiovascular diseases. This is due to a number of mechanisms: For instance, smoking damages the endothelium (the lining of the blood vessels), increases fatty deposits in the arteries, increases clotting, raises low-density lipoprotein cholesterol, reduces high-density lipoprotein and promotes coronary artery spasm. Further, nicotine which is comprised as the addictive component in tobacco accelerates the heart rate and raises blood pressure.

Hyperthyroidism and Related Thyrotoxicosis

Hyperthyroidism is characterized by too high levels of thyroxine. Hyperthyroidism can accelerate a body's metabolism, causing unintentional weight loss and a rapid or irregular heartbeat.

Thyrotoxicosis is a condition characterized by an excess of thyroid hormone in the body.

Chronic Obstructive Pulmonary Disorder (COPD)

“Chronic obstructive pulmonary disease (COPD)” is a disease that is characterized by persistent respiratory symptoms and airflow limitations that are due to airway and/or alveolar abnormalities usually caused by significant exposure to noxious particles or gases. The main risk factor is tobacco smoking but other environmental exposures may contribute.

Cardiomyopathy

Cardiomyopathies are defined by structural and functional abnormalities of the ventricular myocardium that are unexplained by flow limiting coronary artery disease or abnormal loading conditions. Historically, this group of disorders has been subdivided into primary disease, in which the heart is the only involved organ, and secondary forms where the cardiomyopathy is a manifestation of a systemic disorder. ESC Guidelines adopt a classification system in which cardiomyopathies are defined by specific morphological and functional criteria and then grouped into familial/genetic and non-familial/non-genetic subtypes, irrespective of the presence of extra-cardiac disease.

In a particular embodiment of the invention, the cardiomyopathy is a genetic cardiomyopathy or an acquired cardiomyopathy.

Inflammation

There is an association between pro-inflammatory biomarkers and incident hypertension, metabolic syndrome, coronary artery disease (CAD), acute coronary syndrome (ACS), peripheral artery disease, stroke and recurrent coronary and cerebrovascular events. In a particular embodiment the systemic inflammation is associated with an increased C-reactive protein (CRP). CRP is an unspecific marker of inflammation. The range of about 2-3 mg/L is the limit above which there are considered to be signs of systemic inflammation. The role of inflammation in the pathogenesis of atherosclerosis has been firmly established in the past two decades.

Dialysis

A subject on dialysis is a subject that is at an increased risk of a cardiovascular adverse event.

In a particular embodiment of the invention, the dialysis is hemodialysis or peritoneal dialysis.

Medication

There is an increased risk for a cardiovascular adverse event if a subject is treated with one or more of the following:
(i) modulators of the hypoxia-inducible factor (HIF) signaling pathways, including prolyl-hydroxylase inhibitors, such as daprodustat, vadadustat, roxadustat, molidustat, and desidusta;
(ii) erythropoiesis-stimulating agents (ESA), such as Erythropoietin (Epo), Epoetin alfa (Procrit/Epogen), Epoetin beta (NeoRecormon), Darbepoetin alfa (Aranesp), and Methoxy polyethylene glycol-epoetin beta (Mircera); and
(iii) hepcidin modulators such as a hepcidin agonist or a hepcidin antagonist.

Subjects may take one or more erythropoiesis-stimulating agents (ESAs) in an effort to control anemia. ESAs work by helping the body to produce red blood cells. These red blood cells are then released from the bone marrow into the bloodstream where they help maintain blood iron levels. Erythropoiesis-stimulating agents, commonly abbreviated as ESAs, are agents that are similar in structure and/or function to the cytokine erythropoietin, which stimulates red blood cell production (erythropoeisis) in the body. Typical ESAs, structurally and biologically, are similar to naturally occurring protein erythropoietin. Examples of commercially available ESAs include Erythropoietin (Epo), Epoetin alfa (Procrit/Epogen), Epoetin beta (NeoRecormon), Darbepoetin alfa (Aranesp), and Methoxy polyethylene glycol-epoetin beta (Mircera). The two ESAs presently approved for marketing in the U.S. are Epoetin alfa (Procrit, Epogen), and Darbepoietin alfa (Aranesp).

ESAs are commonly given to ESRD subjects. These subjects usually have lower hemoglobin levels because they can't produce enough erythropoietin. The side effects that occur most often with ESA use include: high blood pressure; swelling; fever; dizziness; nausea; and pain at the site of the injection, among others. In addition to these side effects, there are several safety issues that result from ESA use. ESAs increase the risk of venous thromboembolism (blood clots in the veins). ESAs can also cause hemoglobin to rise too high, which puts the subject at higher risk for heart attack, stroke, heart failure, and death.

In a further particular embodiment of the invention, the subject is treated with an anticoagulant and/or an NSAID.

Hereditary Hemorrhagic Telangiectasia

Hereditary hemorrhagic telangiectasia (also known as Osler-Weber-Rendu disease) is a genetic disorder that is inherited from parents. Its severity can vary greatly from person to person, even within the same family. It causes abnormal connections, called arteriovenous malformations (AVMs), to develop between arteries and veins. The most common locations affected are the nose, lungs, brain and liver. These AVMs may enlarge over time and can bleed or rupture, sometimes causing catastrophic complications.

Hereditary Iron Refractory Iron Deficiency Anemia

Hereditary iron-refractory iron deficiency anemia (IRIDA) is an inherited disorder characterized by impaired in absorption and utilization. IRIDA is generally understood to be associated with genetic mutations leading to upregulation of hepcidin. Specific cases of IRIDA are understood to be related to mutations in the gene TMPRSS6. IRIDA is generally refractory to oral iron, but partially responsive to parenteral iron.

FGF23

An elevated level of fibroblast growth factor 23 (FGF23) has come to be understood as a risk factor for cardiovascular disease.

“Fibroblast growth factor 23 (FGF23)” is an osteocyte-derived hormone that regulates phosphate and vitamin D homeostasis. FGF23 undergoes proteolytic cleavage and as a result a mix of uncleaved, i.e. intact FGF23 (iFGF23), and its cleavage fragments are found in vivo. The intact form, iFGF23, is the active form in relation to phosphate metabolism where it controls the urinary excretion of phosphate, with increasing levels of iFGF23 leading to urinary wasting of phosphate. Two main types of antibody assays currently exist, one which captures only iFGF23 and another which binds to the C-terminal end of the hormone and therefore captures both iFGF23 and C-terminal fragments. The latter metric, cFGF23, is therefore a measure of the sum of intact FGF23 and C-terminal FGF23 fragments. Thus, two test related to FGF23 exist, iFGF23 and cFGF23, which have different interpretations.

Elevated FGF23 levels are associated independently with prevalent and incident LVH, cardiovascular disease events, and mortality in CKD and non-CKD populations. Furthermore, a recent study demonstrated that FGF23 directly induces LVH, suggesting that FGF23 is not simply a biomarker of cardiovascular risk. CKD subjects tend to have very high FGF23 levels due to ongoing attempts of the body to compensate for the high serum phosphate levels by producing iFGF23 to increase the urinary fractional excretion of phosphate via the kidneys. For this reason iFGF23 levels are elevated in CKD subjects, and as a result other downstream effects of iFGF23 may be more pronounced.

Increases in FGF23 levels help maintain serum phosphate in the normal range in CKD, FGF23 concentrations increase with decreasing estimated glomerular filtration rate (eGFR) and help maintain normal phosphate homeostasis despite reduced renal mass by stimulating greater per-nephron phosphate excretion and decreasing 1,25-dihydroxyvitamin D levels. Parenteral iron suppresses renal tubular phosphate reabsorption and 1-alpha-hydroxylation of vitamin D resulting in hypophosphatemia. Data indicates that hypophosphatemia is mediated by an increase in FGF23.

Prospective studies in populations of pre-dialysis CKD, incident and prevalent ESRD, and kidney transplant recipients demonstrate that elevated FGF23 levels are independently associated with progression of CKD and development of cardiovascular events and mortality. It was originally thought that these observations were driven by elevated FGF23 levels acting as a highly sensitive biomarker of toxicity due to phosphate. However, FGF23 itself has now been shown to mediate ‘off-target’, direct, end-organ toxicity in the heart, which suggests that elevated FGF23 levels may be a novel mechanism of adverse outcomes in CKD.

In specific embodiments, the subject treated in accordance with the methods described herein for one or more cardiovascular adverse events has elevated FGF23 levels. The normal level of intact FGF23 in the serum of healthy humans is approximately 26.1 pg/mL and the normal level of C-terminal FGF23 fragments in the serum of healthy humans is approximately 49.0 RU/mL. In certain embodiments, the subject's FGF23 levels (e.g., intact FGF23 and/or C-terminal FGF23 fragments) are elevated relative to the normal range in healthy humans. In some embodiments, the subject's intact FGF23 levels in serum are between 250 pg/mL and 350 pg/mL, 200 pg/mL and 300 pg/mL, 200 pg/mL and 350 pg/mL, 300 pg/mL and 350 pg/mL, 300 pg/mL and 400 pg/mL, or 250 pg/mL and 500 pg/mL. In certain embodiments, the subject's intact FGF23 levels in serum are above 200 pg/mL, 225 pg/mL, 250 pg/mL, 275 pg/mL, 300 pg/mL, 325 pg/mL, or 350 pg/mL. In some embodiments, the subject's C-terminal FGF23 fragment levels in serum are between 60 RU/mL and 100 RU/mL, 100 RU/mL and 200 RU/mL, 200 RU/mL and 300 RU/mL, or 250 RU/mL and 400 RU/mL. In certain embodiments, the subject's C-terminal FGF23 fragment levels in serum are above 60 RU/mL, 100 RU/mL, 125 RU/mL, 150 RU/mL, 175 RU/mL, 200 RU/mL, 225 RU/mL, 250 RU/mL, 275 RU/mL, or 300 RU/mL

In some embodiments, subjects treated according to the methods disclosed herein experience an increase in hemoglobin concentration and/or a decrease in FGF23. In specific embodiments, subjects treated according to the methods disclosed herein experience an increase in the hemoglobin level of the subject to a level above 10 g/dL, above 11 g/dL, above 12 g/dL, above 13 g/dL, or above 15 g/dL. In certain embodiments, subjects treated according to the methods disclosed herein experience an increase in the hemoglobin level of the subject to a level between 10 g/dL to 11 g/dL, 11 g/dL to 12 g/dL, 10 g/dL to 13 g/dL, 11 g/dL to 13 g/dL, 11 g/dL to 15 g/dL, or 12 g/dL to 15 g/dL. In some embodiments, subjects treated according to the methods disclosed herein experience a decrease of at least 15%, 20%, 25%, 30%, 35%, 40%, 45%, 50%, 55%, or 60% of intact FGF23 levels in serum or plasma. In certain embodiments, subjects treated according to the methods disclosed herein experience a decrease of 15% to 30%, 20% to 30%, 25% to 50%, 30% to 60% or 15% to 60% of intact FGF23 levels in serum or plasma. In some embodiments, subjects treated according to the methods disclosed herein experience a decrease of at least 15%, 20%, 25%, 30%, 35%, 40%, 45%, 50%, 55%, or 60% of C-terminal FGF23 fragment levels in serum or plasma. In some embodiments, subjects treated according to the methods disclosed herein experience a decrease of 15% to 30%, 20% to 30%, 25% to 50%, 30% to 60% or 15% to 60% of C-terminal FGF23 fragment levels in serum or plasma.

Accordingly, in a first preferred embodiment of this aspect of the invention, a particular group of subjects that are amenable to treatment according to the present invention are subjects having a history of myocardial infarction (MI), a history of stroke, a history of atrial fibrillation (AF), a history of congestive heart failure (CHF), chronic kidney disease (CKD), a history of hypertension, diabetes, a history of heart valve disorder, a history of obesity, and/or being a smoker, a drinker, and/or an elderly subject. These include, in particular, subjects having a history of myocardial infarction (MI), a history of stroke, a history of atrial fibrillation (AF), chronic kidney disease (CKD), a history of congestive heart failure (CHF), and/or a history of hypertension. Subjects having a history of myocardial infarction (MI), a history of stroke, and/or a history of congestive heart failure (CHF) are a particularly preferred group of subjects being at risk of a cardiovascular adverse event and therefore amenable to treatment according to the present invention.

A subject having a history of a particular condition, disorder or disease, such as a history of myocardial infarction, stroke, atrial fibrillation, or congestive heart failure (CHF), is meant to refer to the subject's past and present medical history. The medical history is an account of all medical events and problems a subject has experienced, i.e., a subject's previous medical illnesses, diagnoses, and the patient's general health throughout the life span. A subject having a history of a particular condition, disorder or disease does not necessarily have to have a particular condition, disorder or disease at a given point in time for it to be classified as being at risk. A history of a particular condition, disorder or disease is sufficient. For instance, a subject who has stopped smoking is still at risk because it has a history of smoking.

In a second preferred embodiment of this aspect of the invention, a particular group of subjects that are amenable to treatment according to the present invention are subjects having a history of congestive heart failure (CHF). These include, in particular, subjects also having a history of myocardial infarction (MI) and/or a history of stroke. In a preferred embodiment, CHF is heart failure with reduced ejection fraction (HFrEF).

In a third preferred embodiment of this aspect of the invention, a particular group of subjects that are amenable to treatment according to the present invention are subjects having a history of myocardial infarction (MI). In a preferred embodiment, MI is STEMI or non-STEMI. In a fourth preferred embodiment of this aspect of the invention, a particular group of subjects that are amenable to treatment according to the present invention are subjects having a history of stroke.

Subjects as defined herein (i.e., those being at risk of a cardiovascular adverse event and/or having a history of congestive heart failure) include subjects also having chronic kidney disease (CKD) or subjects not having CKD. Those having CKD are a particular subgroup of patients amenable to treatment according to the present invention. In particular, these include subjects having chronic kidney disease (CKD) and congestive heart failure (CHF), wherein the chronic kidney disease (CKD) is preferably non-dialysis dependent chronic kidney disease (NDD-CKD).

In one embodiment, the subject having a history of congestive heart failure has HFrEF. In another embodiment, the subject having a history of congestive heart failure has congestive heart failure in NYHA class II-IV, in particular HFrEF-type congestive heart failure in NYHA class II-IV. According to another embodiment the subject has congestive heart failure in NYHA class I.

In a number of further embodiments of this aspect of the invention, particular groups of subjects that are amenable to treatment according to the present invention are subjects having one or more of the following risk factors:
(i) subjects having a history of atrial fibrillation (AF), in particular First Diagnosed AF, Proxysmal AF, or Persistent AF;
(ii) subjects having a history of heart valve disorder;
(iii) subjects having a history of hypertension;
(iv) subjects having diabetes;
(v) subjects having a history of obesity;
(vi) elderly subjects, in particular subjects of 60 years or above, 65 year or above, 70 years or above, 75 years or above, or 80 years or above;
(vii) smokers;
(viii) drinkers;
(ix) subjects having hyperthyroidism and/or relatedly thyrotoxicosis;
(x) subjects having chronic obstructive pulmonary disorder (COPD);
(xi) subjects having a cardiomyopathy, in particular a genetic cardiomyopathy or an acquired cardiomyopathy;
(xii) subjects having a systemic inflammation in absence of infection, wherein the systemic inflammation is in particular one that is associated with an increased C-reactive protein (CRP) above the range of about 2-3 mg/L;
(xiii) subjects on dialysis, wherein the dialysis is, in particular, hemodialysis or peritoneal dialysis;
(xiv) subjects treated with one or more of the following:
   a) modulators of the hypoxia-inducible factor (HEF) signaling pathways, including prolyl-hydroxylase inhibitors, such as daprodustat, vadadustat, roxadustat, molidustat, and desidusta;
   b) erythropoiesis-stimulating agents (ESA), such as Erythropoietin (Epo), Epoetin alfa (Procrit/Epogen), Epoetin beta (NeoRecormon), Darbepoetin alfa (Aranesp), and Methoxy polyethylene glycol-epoetin beta (Mircera); and
   c) hepcidin modulators such as a hepcidin agonist or a hepcidin antagonist;
(xv) subjects treated with an anticoagulant and/or an NSAID;
(xvi) subjects having hereditary hemorrhagic telangiectasia; or
(xvii) subjects having hereditary iron refractory iron deficiency anemia.

### II. Therapeutic Benefits

In addition to delivering iron to the subject, the disclosure provides methods for the reduction of the incidence of, or risk for, cardiovascular adverse events in subjects as defined herein. A reference to the reduction of the incidence of, or risk for, “cardiovascular adverse events” or “a cardiovascular adverse event” is meant to indicate that the incidence of, or risk for, one or more of the recited events is reduced.

According to one embodiment of this second aspect of the invention, the cardiovascular adverse event, the incidence of, or risk for, which is reduced, is selected from the group consisting of events that affect the heart (cardiac adverse events); events that affect the peripheral vasculature (peripheral vascular adverse events); events that affect the cerebral vasculature (cerebrovascular adverse events); respiratory, thoracic and mediastinal adverse events; general adverse events; and infections and infestations.

In a particular embodiment of this second aspect of the invention, the cardiac adverse event, the incidence of, or risk for, which is reduced, is selected from the group consisting of congestive heart failure, atrial fibrillation, cardiac arrest, atrioventricular block, cardiac failure, sinus node dysfunction, acute myocardial infarction, bradycardia, angina pectoris, myocardial ischemia, and ventricular extrasystoles; the peripheral vascular adverse events are selected from the group consisting of hypertension, increased systolic blood pressure, increase blood pressure, increased troponin, and hypotension; the cerebrovascular adverse events are selected from the group consisting of cerebrovascular accident, cerebral infarction, and transient ischemic attack; the respiratory, thoracic and mediastinal adverse events are selected from the group consisting of dyspnea and pulmonary edema; the general adverse events are selected from the group consisting of chest pain and death; and the infections and infestations are septic shock.

According to a preferred embodiment of this second aspect of the invention, the cardiovascular adverse event, the incidence of, or risk for, which is reduced, is selected from the group consisting of events that affect the heart (cardiac adverse events); events that affect the peripheral vasculature (peripheral vascular adverse events); events that affect the cerebral vasculature (cerebrovascular adverse events); and death. Preferably, the events that affect the heart (cardiac adverse events) are congestive heart failure, myocardial infarction, unstable angina, and arrhythmia; events that affect the peripheral vasculature (peripheral vascular adverse events) are hypertension; and events that affect the cerebral vasculature (cerebrovascular adverse events) is stroke.

According to a particularly preferred embodiment of this second aspect of the invention, the cardiovascular adverse event, the incidence of, or risk for, which is reduced, is selected from the group consisting of congestive heart failure, myocardial infarction, unstable angina, arrhythmia, hypertension, hypotension, stroke, and death.

According to a further particularly preferred embodiment of this second aspect of the invention, the cardiovascular adverse event, the incidence of, or risk for, which is reduced, is congestive heart failure, atrial fibrillation, hypertension, and/or cardiac arrest.

Specific cardiovascular adverse events related to congestive heart failure are hospitalization, hospitalization due to worsening congestive heart failure or death due to congestive heart failure. The present invention is in particular directed to those CV adverse events where the CV adverse event is CHF.

### III. Therapeutic Benefits in Selected Subjects

Taking the particular groups of subjects as defined herein and the cardiovascular adverse event, the incidence of, or risk for, which is to be reduced into account, the present invention, according to a third aspect, relates to a method of treating iron deficiency in a subject, wherein the treatment of iron deficiency reduces the incidence of, or risk for, a cardiovascular adverse event in the subject, which method comprises administering an effective amount of iron isomaltoside,

wherein the subject is:
(A) a subject being at risk of a cardiovascular adverse event, as defined herein;
(B) a subject having a history of congestive heart failure (CHF); or
(C) a subject having a history of congestive heart failure (CHF) and being at risk of a cardiovascular adverse event, and
   wherein the cardiovascular adverse event the incidence of, or risk for, which reduced is:
(a) selected from the group consisting of congestive heart failure, myocardial infarction, unstable angina, arrhythmia, hypertension, hypotension, stroke, and death;
(b) congestive heart failure;
(c) atrial fibrillation;
(d) hypertension; or
(e) cardiac arrest.

According to a preferred embodiment of this third aspect of the invention, the subject is a subject being at risk of a cardiovascular adverse event and the cardiovascular adverse event, the incidence of, or risk for, which is reduced, is congestive heart failure.

According to a further preferred embodiment of this third aspect of the invention, the subject is a subject being at risk of a cardiovascular adverse event and the cardiovascular adverse event the incidence of, or risk for, which is reduced, is atrial fibrillation.

According to a further preferred embodiment of this third aspect of the invention, the subject is a subject being at risk of a cardiovascular adverse event and the cardiovascular adverse event the incidence of, or risk for, which is reduced, is cardiac arrest.

According to a further preferred embodiment of this third aspect of the invention, the subject is a subject having a history of congestive heart failure (CHF) and the cardiovascular adverse event the incidence of, or risk for, which is reduced, is cardiac arrest or congestive heart failure or both.

According to a further preferred embodiment of this third aspect of the invention, the subject is a subject having a history of congestive heart failure (CHF) and the cardiovascular adverse event the incidence of, or risk for, which is reduced, is congestive heart failure.

According to a further preferred embodiment of this third aspect of the invention, the subject is a subject having a history of congestive heart failure (CHF) and the cardiovascular adverse event the incidence of, or risk for, which is reduced, is atrial fibrillation.

According to a further preferred embodiment of this third aspect of the invention, the subject is a subject having a history of congestive heart failure (CHF) and the cardiovascular adverse event the incidence of, or risk for, which is reduced, is cardiac arrest.

According to another embodiment of this third aspect of the invention, the subject is a subject being at risk of a cardiovascular adverse and/or having a history of congestive heart failure which has chronic kidney disease (CKD). According to another embodiment of this third aspect of the invention, the subject is a subject being at risk of a cardiovascular adverse and/or having a history of congestive heart failure which does not have chronic kidney disease (CKD). In particular, these include subjects having chronic kidney disease (CKD) and/or congestive heart failure (CHF), wherein the chronic kidney disease (CKD) is preferably non-dialysis dependent chronic kidney disease (NDD-CKD).

In some embodiments, subjects having a history of congestive heart failure have HFrEF. In some embodiments, subjects having a history of congestive heart failure have congestive heart failure in NYHA class of II-IV, in particular HFrEF-type congestive heart failure in NYHA class II-IV. According to another embodiment the subject has congestive heart failure in NYHA class I.

In other embodiments, the disclosure provides methods for the reduction of the incidence of, or risk for, hospitalizations related to cardiovascular adverse events in subjects as defined herein.

In some embodiments, the disclosure provides methods of reducing mortality and morbidity, i.e., in particular death, due to cardiovascular adverse events in subjects as defined herein.

### IV. Management of Atrial Fibrillation

The invention is further based on the finding that iron therapy effectively reduces P-wave dispersion. As prolongation of atrial electrical conduction, reflected by an increased P-wave dispersion/duration, is a hallmark feature in those who go on to develop and remain in AF, the reduction of P-wave dispersion/duration is critical for the prevention and treatment of AF. Accordingly, in one embodiment the present invention provides a method for the reduction of P-wave dispersion/duration for the prevention or treatment of AF or disorders associated with AF (e.g., heart failure, hypertension, heart valve disease, coronary artery disease, obesity, and diabetes mellitus) in an animal suffering from such a condition which comprises administering to such an animal a therapeutically effective amount of an iron agent.

Preferably, the subject for treatment will be a human. However, the treatment may be a veterinary one such as in horses or cats.

The method of the invention may be used to prevent and treat AF, including paroxysmal, persistent, long-standing, and chronic AF.

By “prevent” in the context of AF we mean stopping the first episode or repeated occurrences of AF in an animal that is in normal sinus rhythm at the time of iron therapy. By “treat” in the context of AF we mean conversion of AF into a normal sinus rhythm. Prevention and treatment of AF would ultimately result in a reduction in the burden of AF which should translate to reduce symptoms (e.g., palpitations, breathlessness), exercise intolerance, hospitalization, heart failure, stroke and death.

Iron agents are well known and may include oral tablets (e.g., ferrous sulphate, ferrous fumarate, ferrous gluconate, iron polysaccharide, iron polymaltose), intramuscular injections, or intravenous injections/infusions (e.g., high molecular weight iron dextran, low molecular weight iron dextran, iron sucrose, ferrous gluconate, ferric carboxymaltose, iron isomaltoside).

It is also possible in accordance with the invention for the iron agent to be used in animals who may or may not be iron deficient on blood testing as many patients have myocardial iron deficiency that responds to supplementation despite having normal blood tests for iron deficiency.

### V. Dosing and Administration Regimens

The methods of treating iron deficiency in a subject being at risk of a cardiovascular adverse event according to the invention comprise administering an effective amount of iron isomaltoside.

A typical treatment regimen of iron isomaltoside would consist either of a single infusion of 1000 mg of iron, a dose of up to 20 mg iron/kg body weight of elemental iron given as intravenous infusion, or as an intravenous bolus injection up to 500 mg up to three times a week. The cumulative iron need can be determined using the Ganzoni formula and according to one embodiment, the calculated dose will be administered. Alternatively, the cumulative dose that will be administered is chosen according to the following Table:

| | Patients with body weight | Patients with body weight |
|---|---|---|
| Hb (g/dL) | 50 kg to <70 kg | ≥70 kg |
| ≥10 | 1000 mg | 1500 mg |
| <10 | 1500 mg | 2000 mg |

Alternatively, a typical treatment regimen of iron isomaltoside would be selected according to the following Table:

| | Body weight | Body weight | Body weight |
|---|---|---|---|
| Hb (g/dL) | <50 kg | 50 to <70 kg | ≥70 kg |
| >10 | 20 mg/kg | 1000 mg | 20 mg/kg |
| | | | up to a maximum of 1500 mg |
| <10 | 20 mg/kg | 20 mg/kg | 20 mg/kg |
| | | | up to a maximum of 2000 mg |

Therefore, in some embodiments, an effective amount of iron isomaltoside is an amount ranging from about 500 mg to 2000 mg, e.g. at 500 mg, 1000 mg, 1500 mg or 2000 mg elemental iron, which can be administered in a single dose or in more than one, in particular 2 or 3, doses. Alternatively, an effective amount of iron isomaltoside is an amount of up to 50 mg iron/kg body weight, in particular up to 30 mg iron/kg body weight, or preferably up to 20 mg iron/kg body weight.

In a particular embodiment, the dose is a single daily dose. For instance, a typical single daily dose of iron isomaltoside is 1000 mg elemental iron.

For repeated administration, a first dose of from 200 to 700 mg, preferably from 300 to 600, most preferably of up to 500 mg elemental iron is followed by a second dose of from 200 to 700 mg, preferably from 300 to 600, and most preferably of up to 500 elemental iron. The two doses may be administered within 1 month, 2 weeks, or preferably 1 week. Preferably, they are administered within one week. Further doses of iron isomaltoside may follow, for instance a third dose of from 200 to 700 mg, preferably from 300 to 600, most preferably of up to 500 mg elemental iron. This further, e.g. third, dose(s) may be administered within the same time frame, i.e., 1 month, 2 weeks, or preferably 1 week. These multiple doses are preferably administered as a bolus injection. It is further preferred if each dose is administered at least 2 and in particular 3 days apart. For instance, if 3 doses are to be administered within one week, it is preferred to administer these doses on day 1, 4 and 7.

When ferric bepectate is used as the iron carbohydrate complex of the invention, the calculation of the effective cumulative dose as elemental iron can likewise be determined based on the Ganzoni formula or the tables provided above. Alternatively, 1000 mg or 1500 mg of elemental iron can be an effective cumulative dose. Appropriate single doses of ferric bepectate, i.e., the dose administered in one sitting, are for example 500 mg, 1000 mg, or 1500 mg up to 15 mg/kg or alternatively up to 20 mg/kg. Alternatively, the appropriate single dose can be 15 mg/kg or 20 mg/kg body weight.

For CKD patients on hemodialysis, repeat doses of iron isomaltoside or ferric bepectate may be administered in conjunction with dialysis sessions, such as, for example, 100 mg to 500 mg, preferably 100 mg or 200 mg.

### C. Drug Combinations

Further described herein are combinations of iron isomaltoside with one or more additional drugs for use in treating iron deficiency according to the invention, wherein the additional drug is selected from the group consisting of:
(1) an angiotensin-converting enzyme inhibitor (ACEI), such as captopril, enalapril, lisinopril, ramipril, or trandolapril;
(2) a beta-blocker, such as bisoprolol, carvedilol, metoprolol succinate, or nebivolol;
(3) a mineralocorticoid receptor antagonist (MRA), such as eplerenone or spironolactone;
(4) an angiotensin receptor blocker, such as candesartan, valsartan, or losartan;
(5) an If-channel blocker, such as ivabradine;
(6) an angiotensin receptor neprilysin inhibitor, such as sacubitril/valsartan; and
(7) a diuretic, such as furosemide, bumetanide, torasemide, bendroflumethoazide, hydrochlorothiazide, metolazone, indapamide, amiloride, or triamterene.

Also described are combinations of iron isomaltoside with one or more additional drugs for use in treating iron deficiency according to the invention, wherein the additional drug is a cardiac glycoside, such as digoxin or digitoxin.

Dosages for the additional drugs usually refer to amounts of drug administered to adults. Dosages for administration to infants may be adjusted accordingly.

Particular combinations of iron isomaltoside with one or more additional drugs for use in treating iron deficiency according to the invention are wherein the additional drug puts the subject at higher risk for a cardiovascular adverse event. According to this aspect, the additional drug is selected from the group consisting of:
(i) modulators of the hypoxia-inducible factor (HIF) signaling pathways, including prolyl-hydroxylase inhibitors, such as daprodustat, vadadustat, roxadustat, molidustat, and desidusta;
(ii) erythropoiesis-stimulating agents (ESA), such as Erythropoietin (Epo), Epoetin alfa (Procrit/Epogen), Epoetin beta (NeoRecormon), Darbepoetin alfa (Aranesp), and Methoxy polyethylene glycol-epoetin beta (Mircera); and
(iii) hepcidin modulators such as a hepcidin agonist or a hepcidin antagonist.

### Exemplary Embodiments

1. A method of treating iron deficiency in a subject being at risk of a cardiovascular adverse event, which comprises administering an effective amount of iron isomaltoside.
2. The method of embodiment 1 wherein the subject being at risk of a cardiovascular adverse event is a subject having a history of myocardial infarction (MI), a history of stroke, a history of atrial fibrillation (AF), a history of congestive heart failure (CHF), chronic kidney disease (CKD), a history of hypertension, diabetes, a history of heart valve disorders, a history of obesity, and/or being a smoker, a drinker, and/or an elderly subject.
3. The method of embodiment 2 wherein the subject being at risk of a cardiovascular adverse event is a subject having a history of myocardial infarction (MI), a history of stroke, a history of atrial fibrillation (AF), chronic kidney disease (CKD), a history of congestive heart failure (CHF), and/or a history of hypertension.
4. The method of embodiment 3, wherein the subject being at risk of a cardiovascular adverse event is a subject having a history of myocardial infarction (MI), a history of stroke, and/or a history of congestive heart failure (CHF).
5. A method of treating iron deficiency in a subject having a history of congestive heart failure (CHF), which comprises administering an effective amount of iron isomaltoside.
6. The method of embodiment 5, wherein the subject having a history of congestive heart failure (CHF) has a history of myocardial infarction (MI) and/or a history of stroke.
7. The method of any one of embodiments 2-6, wherein the congestive heart failure (CHF) is heart failure with reduced ejection fraction (HFrEF).
8. The method of embodiments 1-4, wherein the subject being at risk of a cardiovascular adverse event is a subject having a history of myocardial infarction (MI).
9. The method of embodiment 8, wherein the myocardial infarction (MI) is STEMI or non-STEMI.
10. The method of embodiments 1-4, wherein the subject being at risk of a cardiovascular adverse event is a subject having a history of stroke.
11. The method of any one of embodiments 1-7, wherein the subject is a subject being at risk of a cardiovascular adverse event and/or having a history of congestive heart failure, and wherein the subject has chronic kidney disease (CKD).
12. The method of any one of embodiments 1-7, wherein the subject is a subject being at risk of a cardiovascular adverse event and/or having congestive heart failure, and wherein the subject does not have chronic kidney disease (CKD).
13. The method of any one of embodiments 1-11, wherein the subject being at risk of a cardiovascular adverse event is a subject having chronic kidney disease (CKD) and a history of congestive heart failure (CHF).
14. The method of embodiment 13, wherein the subject having a history of congestive heart failure has HFrEF.
15. The method of embodiment 13 or 14, wherein the subject having a history of congestive heart failure has congestive heart failure in New York Heart Association (NYHA) class II-IV.
15A. The method of embodiment 13 or 14, wherein the subject has congestive heart failure in New York Heart Association (NYHA) class I.
16. The method of any one of embodiments 11-15 or 15A, wherein the chronic kidney disease (CKD) is non-dialysis dependent chronic kidney disease (NDD-CKD).
17. The method of embodiments 1-3, wherein the subject being at risk of a cardiovascular adverse event is a subject having a history of atrial fibrillation (AF).
18. The method of embodiment 17, wherein atrial fibrillation (AF) is First Diagnosed AF, Proxysmal AF, or Persistent AF.
19. The method of embodiment 1 or 2, wherein the subject being at risk of a cardiovascular adverse event is a subject having a history of heart valve disorders.
20. The method of embodiments 1-3, wherein the subject being at risk of a cardiovascular adverse event is a subject having a history of hypertension.
21. The method of embodiment 1 or 2, wherein the subject being at risk of a cardiovascular adverse event is a subject having diabetes.
22. The method of embodiment 1 or 2, wherein the subject being at risk of a cardiovascular adverse event has a history of obesity.
23. The method of embodiment 1 or 2, wherein the subject being at risk of a cardiovascular adverse event is an elderly subject, a smoker, or a drinker.
24. The method of embodiment 23, wherein the elderly subject is 60 years or above, 65 year or above, 70 years or above, 75 years or above, or 80 years or above.
25. The method of embodiment 1, wherein the subject being at risk of a cardiovascular adverse event is a subject having hyperthyroidism and/or relatedly thyrotoxicosis.
26. The method of embodiment 1, wherein the subject being at risk of a cardiovascular adverse event is a subject having chronic obstructive pulmonary disorder (COPD).
27. The method of embodiment 1, wherein the subject being at risk of a cardiovascular adverse event is a subject having a cardiomyopathy.
28. The method of embodiment 27, wherein the cardiomyopathy is a genetic cardiomyopathy or an acquired cardiomyopathy.
29. The method of embodiment 1, wherein the subject being at risk of a cardiovascular adverse event is a subject having a systemic inflammation in absence of infection.
30. The method of embodiment 29, wherein the systemic inflammation is associated with an increased C-reactive protein (CRP) above the range of about 2-3 mg/L.
31. The method of embodiment 1, wherein the subject being at risk of a cardiovascular adverse event is a subject on dialysis.
32. The method of embodiment 31, wherein the dialysis is hemodialysis or peritoneal dialysis.
33. The method of embodiment 1, wherein the subject being at risk of a cardiovascular adverse event is a subject treated with one or more of the following:
a) modulators of the hypoxia-inducible factor (HEF) signaling pathways, including prolyl-hydroxylase inhibitors, such as daprodustat, vadadustat, roxadustat, molidustat, and desidusta;
b) erythropoiesis-stimulating agents (ESA), such as Erythropoietin (Epo), Epoetin alfa (Procrit/Epogen), Epoetin beta (NeoRecormon), Darbepoetin alfa (Aranesp), and Methoxy polyethylene glycol-epoetin beta (Mircera); and
c) hepcidin modulators such as a hepcidin agonist or a hepcidin antagonist.
   34. The method of embodiment 1, wherein the subject being at risk of a cardiovascular adverse event is a subject treated with an anticoagulant and/or an NSAID.
   35. The method of embodiment 1, wherein the subject being at risk of a cardiovascular adverse event is a subject having hereditary hemorrhagic telangiectasia.
   36. The method of embodiment 1, wherein the subject being at risk of a cardiovascular adverse event is a subject having hereditary iron refractory iron deficiency anemia.
   37. The method of any one of embodiments 1-36, wherein iron deficiency is iron-deficiency anemia.
   38. The method of any one of embodiments 1-37, wherein the subject has chronic iron loss or malabsorption.
   39. The method of any one of embodiments 1-38, wherein the subject does not tolerate oral iron or for whom oral iron is not effective.
   40. The method of any one of embodiments 1-39, wherein the iron deficiency is defined as TSAT <20% and/or ferritin <100 μg/L.
   41. The method of any one of embodiments 1-40, wherein the treatment of iron deficiency reduces the incidence of, or risk for, a cardiovascular adverse event in the subject.
   42. The method of embodiment 41, wherein the cardiovascular adverse event is selected from the group consisting of events that affect the heart (cardiac adverse events); events that affect the peripheral vasculature (peripheral vascular adverse events); events that affect the cerebral vasculature (cerebrovascular adverse events); respiratory, thoracic and mediastinal adverse events; general adverse events; and infections and infestations.
   43. The method of embodiment 42, wherein the cardiac adverse events are selected from the group consisting of congestive heart failure, atrial fibrillation, cardiac arrest, atrioventricular block, cardiac failure, sinus node dysfunction, acute myocardial infarction, bradycardia, angina pectoris, myocardial ischemia, and ventricular extrasystoles; the peripheral vascular adverse events are selected from the group consisting of hypertension, increased systolic blood pressure, increase blood pressure, increased troponin, and hypotension; the cerebrovascular adverse events are selected from the group consisting of cerebrovascular accident, cerebral infarction, and transient ischemic attack; the respiratory, thoracic and mediastinal adverse events are selected from the group consisting of dyspnea and pulmonary edema; the general adverse events are selected from the group consisting of chest pain and death; and the infections and infestations are septic shock.
   44. The method of embodiment 41, wherein cardiovascular adverse event is selected from the group consisting of events that affect the heart (cardiac adverse events); events that affect the peripheral vasculature (peripheral vascular adverse events); events that affect the cerebral vasculature (cerebrovascular adverse events); and death.
   45. The method of embodiment 44, wherein the events that affect the heart (cardiac adverse events) are congestive heart failure, myocardial infarction, unstable angina, and arrhythmia; events that affect the peripheral vasculature (peripheral vascular adverse events) are hypertension; and events that affect the cerebral vasculature (cerebrovascular adverse events) is stroke.
   46. The method of embodiment 41, wherein the cardiovascular adverse events are selected from the group consisting of congestive heart failure, myocardial infarction, unstable angina, arrhythmia, hypertension, hypotension, stroke, and death.
   47. The method of embodiments 41-42, wherein the cardiovascular adverse event is selected from the group consisting of congestive heart failure, atrial fibrillation, hypertension, and cardiac arrest.
   48. The method of any one of embodiments 41-47, wherein the cardiovascular adverse event is congestive heart failure.
   49. The method of embodiment 41, wherein the cardiovascular adverse event is hospitalization or death due to congestive heart failure.
   50. The method of embodiment 49, wherein the cardiovascular adverse event is hospitalization due to congestive heart failure.
   51. The method of embodiment 49, wherein the cardiovascular adverse event is death due to congestive heart failure.
   52. The method of embodiment 41, wherein the cardiovascular adverse event is cardiovascular death and/or hospitalization due to worsening congestive heart failure.
   53. The method of any one of embodiments 41-43 and 47, wherein the cardiovascular adverse event is atrial fibrillation.
   54. The method of embodiments 41-47, wherein the cardiovascular adverse event is hypertension.
   55. The method of any one of embodiments 41-43 and 47, wherein the cardiovascular adverse event is cardiac arrest.
   56. The method of any one of embodiments 41-55, wherein the iron isomaltoside is ferric derisomaltose.
   57. A method of treating iron deficiency in a subject, wherein the treatment of iron deficiency reduces the incidence of, or risk for, a cardiovascular adverse event in the subject, which method comprises administering an effective amount of iron isomaltoside,
   wherein the subject is:
(A) a subject being at risk of a cardiovascular adverse event;
(B) a subject having a history of congestive heart failure (CHF); or
(C) a subject having a history of congestive heart failure (CHF) and being at risk of a cardiovascular adverse event, and
   wherein the cardiovascular adverse event, the incidence of, or risk for, which is reduced, is:
(a) selected from the group consisting of congestive heart failure, myocardial infarction, unstable angina, arrhythmia, hypertension, hypotension, stroke, and death;
(b) congestive heart failure;
(c) atrial fibrillation;
(d) hypertension; or
(e) cardiac arrest.
   58. The method of embodiment 57, wherein the subject is a subject being at risk of a cardiovascular adverse event and the cardiovascular adverse event, the incidence of, or risk for, which is reduced, is congestive heart failure.
   59. The method of embodiment 57, wherein the subject is a subject being at risk of a cardiovascular adverse event and the cardiovascular adverse event, the incidence of, or risk for, which is reduced, is atrial fibrillation.
   60. The method of embodiment 57, wherein the subject is a subject being at risk of a cardiovascular adverse event and the cardiovascular adverse event, the incidence of, or risk for, which is reduced, is cardiac arrest.
   61. The method of embodiment 57, wherein the subject is a subject having a history of congestive heart failure and the cardiovascular adverse event, the incidence of, or risk for, which is reduced, is cardiac arrest or congestive heart failure or both.
   62. The method of embodiment 57, wherein the subject is a subject having a history of congestive heart failure (CHF) and the cardiovascular adverse event, the incidence of, or risk for, which is reduced, is congestive heart failure.
   63. The method of embodiment 57, wherein the subject is a subject having a history of congestive heart failure (CHF) and the cardiovascular adverse event, the incidence of, or risk for, which is reduced, is atrial fibrillation.
   64. The method of embodiment 57, wherein the subject is a subject having a history of congestive heart failure (CHF) and the cardiovascular adverse event, the incidence of, or risk for, which is reduced, is cardiac arrest.
   65. The method of embodiment 57, wherein the subject is a subject being at risk of a cardiovascular adverse event and/or having a history of congestive heart failure, and wherein the subject has chronic kidney disease (CKD).
   66. The method of embodiment 57, wherein the subject is a subject being at risk of a cardiovascular adverse event and/or having congestive heart failure, and wherein the subject does not have chronic kidney disease (CKD).
   67. The method of embodiment 65 or 66, wherein the chronic kidney disease (CKD) is non-dialysis dependent chronic kidney disease (NDD-CKD).
   68. The method of any one of embodiments 57, or 61-67, wherein the subject having a history of congestive heart failure has HFrEF.
   69. The method of any one of embodiments 57, or 61-68, wherein the subject having a history of congestive heart failure has congestive heart failure in NYHA class II-IV.
   69A. The method of any one of embodiments 57, or 61-68, wherein the subject has congestive heart failure in NYHA class I.
   70. The method of embodiment 57-69 or 69A, wherein the cardiovascular adverse event, the incidence of, or risk for, which is reduced, is cardiovascular death and/or hospitalization due to worsening congestive heart failure.
   71. The method of embodiment 57, wherein the subject being at risk of a cardiovascular adverse event is a subject having chronic kidney disease (CKD) and a history of congestive heart failure (CHF).
   72. The method of any one of embodiments 41-71, wherein iron deficiency is iron-deficiency anemia.
   73. The method of any one of embodiments 41-72, wherein the subject has chronic iron loss or malabsorption.
   74. The method of any one of embodiments 41-73, wherein the subject does not tolerate oral iron or for whom oral iron is not effective.
   75. The method of any one of embodiments 41-74, wherein the iron deficiency is defined as TSAT <20% and/or ferritin <100 μg/L.
   76. The method of any one of embodiments 57-75, wherein the iron isomaltoside is ferric derisomaltose.
   77. The method of any one of embodiments 1-76, further comprising, prior to said administration of iron isomaltoside, determining whether said patient is at risk of a cardiovascular adverse event.
   78. The method of claim 77, wherein said determining comprises a stress test, measuring one or more cardiac enzyme markers, electrocardiography, ambulatory electrocardiography, imaging (such as chest X-ray, echocardiography, tomography, CT, CAT scan, EBCT, PET, DCA, DSA, Multidetector CT, MRI or SPECT), Myocardial Perfusion Imaging (MPI), Multigated Acquisition (MUGA) Scan, radionuclide stress test, nuclear stress test, or coronary angiography.
   79. The method of claim 77 or 78, wherein said iron isomaltoside is administered if said patient is at risk of a cardiovascular adverse event.

The present disclosure is further illustrated by the following examples, which should not be construed as further limiting. The contents of all figures and all references, Genbank sequences, patents and published patent applications cited throughout this application are expressly incorporated herein by reference.

### EXAMPLES

### Example 1

Two Phase III randomized, open-label, comparative safety and efficacy studies were performed in relation to treatment with iron isomaltoside (“IIM”, tradename Monofer®, Monoferric®) and iron sucrose (“IS”, trade name Venofer®) in adult human subjects with iron deficiency anaemia and non-dialysis-dependent chronic kidney disease. These studies allowed comparing the incidence of treatment-emergent cardiovascular adverse events in particular in patients with cardiovascular risk factors.

### Study Design

The studies were randomized, open-label, comparative studies. Subjects with iron deficiency anaemia (IDA) were randomized 1:1 to one treatment course of one of two treatments. Iron isomaltoside 1000 was administered as a single dose of 1000 mg of elemental iron. Iron sucrose was administered as 200 mg slow intravenous bolus injections according to its US label and repeated up to 5 times to reach a cumulative dose of 1000 mg.

### Objectives

The study allowed comparing the incidence of protocol defined cardiovascular adverse events in subjects with IDA and non-dialysis-dependent chronic kidney disease when treated with iron isomaltoside or iron sucrose.

Said protocol defined cardiovascular adverse events were as follows:
(1) Congestive heart failure requiring hospitalization or medical intervention:
   Congestive heart failure requiring hospitalization or medical intervention was defined as meeting the following criteria:
Requires hospitalization defined as an admission to an inpatient unit or a visit to an emergency department that results in at least a 12-hour stay (or a date change if the time of admission/discharge is not available); AND
Clinical manifestation of congestive heart failure including at least one of the following: new or worsening dyspnoea, orthopnoea, paroxysmal nocturnal dyspnoea, oedema, pulmonary basilar crackles, jugular venous distension, or radiological evidence of worsening heart failure; AND
Additional/increased therapy:
   Intravenous treatment with diuretic, inotrope, or vasodilator therapy; OR
   Mechanical or surgical intervention (mechanical circulatory support, heart transplantation, or ventricular pacing to improve cardiac function) or the use of ultrafiltration, hemofiltration, or dialysis that is specifically directed at treatment of heart failure.
(2) Arrhythmia:
   Arrhythmia was defined as any symptomatic deviation from normal sinus rhythm experienced by the subject that results in an evaluation by a health care provider. The evaluation included a physical examination during an outpatient visit, an ECG, or a hospital admission. Arrhythmias included any conduction abnormality, atrioventricular heart block, prolongation of QTc interval, supraventricular/nodal arrhythmia, vasovagal episode, ventricular arrhythmia, or other cardiovascular arrhythmia.
(3) Hypertension
   During the observation period immediately following trial drug administration, hypertension was defined as an increase in systolic blood pressure >20 mm Hg that results in a value >180 mm Hg or an increase in diastolic blood pressure >15 mm Hg that results in a value >105 mm Hg.
   Following the release of a subject from the trial visit during which they are receiving medication, hypertension was defined as requiring an unscheduled outpatient health care visit, a hospital admission, or a change in medical therapy (e.g. administration of antihypertensives) in conjunction with the objective criteria, a rise in blood pressure (an increase in systolic blood pressure >20 mm Hg that results in a value >180 mm Hg or an increase in diastolic blood pressure >15 mm Hg that results in a value >105 mm Hg).
(4) Hypotension
   During the observation period immediately following trial drug administration, hypotension was defined as a decrease in systolic blood pressure >20 mm Hg that results in a value <90 mm Hg or a decrease in diastolic blood pressure >15 mm Hg that results in a value <50 mm Hg.
   Following the release of a subject from the trial visit during which they are receiving medication, hypotension was defined as requiring an unscheduled outpatient health care visit, a hospital admission, or a change in medical therapy (e.g. fluid/volume repletion, holding of antihypertensives) in conjunction with the objective criteria, a decrement in blood pressure (a decrease in systolic blood pressure >20 mm Hg that results in a value <90 mm Hg or a decrease in diastolic blood pressure >15 mm Hg that results in a value <50 mm Hg).
(5) Myocardial infarction (MI):
   A myocardial infarction (MI) was defined as the presence of the characteristic changes in cardiac enzyme markers in the setting of either temporally related symptoms of an acute coronary syndrome or electrocardiographic (ECG) changes consistent with either ischemia or infarction.
   Cardiac enzyme markers indicative of an MI included:
An appropriate rise and fall in serum troponin (I or T) or creatine kinase-MB where at least 1 value is ≥2× upper limit of normal (ULN). Where only 1 value has been measured, if it is ≥2×ULN, an event was adjudicated based on the totality of the clinical evidence.
Where only total creatine phosphokinase has been measured, serial changes (i.e. at least 2 values) needed to be ≥2×ULN.
   Symptoms indicative of ischemia needed to have been present for ≥10 minutes and included chest pain, chest pressure, or chest tightness. Dyspnoea, diaphoresis, or nausea was considered symptoms of ischemia and was judged based on the totality of the clinical evidence.
   ECG changes was defined as:
New Q waves in 2 or more contiguous leads;
Evolving ST-segment to T-wave changes in 2 or more contiguous leads+(such as ≥0.5 mm transient ST-segment depression);
New left bundle branch block; OR
1 mm ST-segment elevation in 2 or more contiguous leads.
(6) Stroke:
   A stroke was defined as a focal neurological deficit of sudden onset that is not reversible within 24 hours that results from a vascular cause involving the central nervous system and is not due to another readily identifiable cause (i.e. brain tumour or trauma). Strokes were sub-classified as hemorrhagic, ischaemic, or unknown.
(7) Unstable angina requiring hospitalization:
   Unstable angina requiring hospitalization was defined as ischemic symptoms meeting the following criteria:
Lasting ≥10 minutes and considered to be myocardial ischemia on final diagnosis; AND
Requiring an unscheduled visit to a health care facility and overnight admission (does not include chest pain observation units); AND
At least one of the following:
   New dynamic ECG changes,
   Ischemia evidence on stress testing with or without cardiac imaging,
   Angiographic evidence of ≥70% lesion and/or thrombus in an epicardial coronary artery.
(8) Death due to any cause:
   Death due to any cause was adjudicated as the date the subject is pronounced dead.

The secondary efficacy objective of the study was to compare the effects of iron isomaltoside and ferric carboxymaltose treatment in subjects with IDA on hemoglobin (Hb), s-ferritin, and transferrin saturation (TSAT).

### Endpoints

The primary safety outcome measure was the incidence of protocol defined hypersensitivity reactions (number of participants with such events) within 8 weeks.

The primary efficacy outcome measure was the ability to increase Hb (g/dL) within 8 weeks.

The secondary safety outcome measure was the incidence of protocol defined cardiovascular adverse events (number of participants with such events) within 8 weeks.

The secondary efficacy outcome measures were changes in s-ferritin (ng/mL) within 8 weeks and changes in transferrin saturation (%) within 8 weeks.

### Safety Assessments

The study included the following safety assessments:
AEs data will be collected and evaluated for relatedness, severity, seriousness, and expectedness. They will be reported to authorities and followed-up according to international and local requirements.
Physical examinations, measurements of vital signs, ECG, height, weight, and safety laboratory parameters.

### Efficacy Assessments

The study included the following efficacy assessments:
Hb, s-ferritin, TSAT (s-iron and transferrin)

### Study Duration and Number of Visits

For the individual subject, duration of the study was 8 weeks (including a 28 days screening period) and each subject attended 6-8 visits.

### Subject Population

Subjects, who fulfilled the following eligibility criteria, were included:
1. Men or women >18 years;
2. Hb≤11 g/dL; and
3. Willingness to participate and signing the Informed Consent Form (ICF).

For IDA-03

Additional inclusion criteria for the IDA-03 study were:
1. IDA caused by different aetiologies* such as abnormal uterine bleeding, gastrointestinal diseases, cancer, bariatric procedures (gastric bypass operations), and other conditions leading to significant blood loss;
2. TSAT <20%;
3. S-ferritin ≤100 ng/mL; and
4. Documented history of intolerance or unresponsiveness to oral iron therapy** for at least one month*** prior to study enrolment.

The mean age of patients was 44 years (range 18-91) and 89% were woman.

For CKD-04

Additional inclusion criteria for the CKD-04 study were:
1. Chronic renal impairment, as defined by either (i) eGFR <60 mL/min/1.73 m² at screening (as calculated by modification of diet in renal disease (MDRD)), or (ii) eGFR <90 mL/min/1.73 m² at screening and kidney damage as indicated by abnormalities in urine composition per medical history and/or intermediate/high risk of cardio-vascular disease based on the Framingham model;
2. Screening s-ferritin ≤100 ng/mL, or ≤300 ng/mL if TSAT ≤30%; and
3. Either no ESAs or ESAs as a stable dose (+/−20%) for 4 weeks before randomisation.
   The mean age of patients was 69 years (range 25-97) and 63% were female.
   *The etiology (also if unknown) for IDA was documented in the medical history and verified in the source documents.
   **The intolerance and non-response to oral iron treatment was documented with sign and symptoms in the medical history and verified in the source document.
   ***There was a documentation of intolerance or unresponsiveness to at least one month of prescribed oral iron therapy per investigator's judgment within the last 9 months and they would not be candidates for oral iron again.

A subject was not eligible for inclusion in this study if he/she fulfilled any of the following criteria:
1. Anaemia predominantly caused by factors other than IDA according to Investigator's judgment
2. Hemochromatosis or other iron storage disorders
3. Previous serious hypersensitivity reactions to any intravenous iron compounds
4. Treatment with intravenous iron within the last 30 days prior to screening
5. Treatment with erythropoietin or erythropoietin-stimulation agents, red blood cell transfusion, radiotherapy, and/or chemotherapy within the last 30 days prior to screening
6. Planned surgical procedure during the study period
7. Alanine Aminotransferase (ALAT) and/or Aspartate Aminotransferase (ASAT) >3 times upper limit of normal (e.g. decompensated liver cirrhosis or active hepatitis)
8. Surgery under general anesthesia within the last 30 days prior to screening
9. Decompensated liver cirrhosis or active hepatitis (only for CKD-04)
10. Required dialysis for treatment of CKD
11. Alcohol or drug abuse within the past 6 months
12. Pregnant or nursing women. In order to avoid pregnancy, women of childbearing potential have to use adequate contraception (e.g. intrauterine devices, hormonal contraceptives, or double barrier method) during the whole study period and 7 days after the last dosing

### Study Treatment

The subjects were dosed with either one treatment course of iron isomaltoside (group A) or one treatment course of iron sucrose (group B) as described below.
Group A: iron isomaltoside was administered as a single intravenous infusion of 1000 mg at baseline diluted in 100 mL 0.9% sodium chloride and given over approximately 20 minutes (50 mg iron/min, cumulative dose: 1000 mg).
Group B: iron sucrose was administered as 200 mg slow intravenous bolus injections according to label and repeated up to 5 times to reach a cumulative dose of 1000 mg.

No premedication (e.g. antihistamine or steroids) was allowed before administration of the study drug. If the subject was in daily treatment for e.g. allergy or asthma this was not considered as “premedication” and participating in the study could be continued.

### Statistical Analyses

The co-primary safety endpoint was analyzed by constructing an exact two-sided 95% CI of the incidence of treatment-emergent serious and/or severe non-serious hypersensitivity AEs in the iron isomaltoside treatment group. If the upper bound of the 95% CI is <3%, the safety objective has been met.

In addition, the risk difference between iron isomaltoside and iron sucrose was assessed by constructing a 95% CI of the risk difference. Both an unadjusted CI (with continuity correction), and a 95% Newcombe CI adjusted for strata using the Cochran-Mantel-Haenszel method will be produced.

As to sensitivity, the treatment groups were compared between the treatment groups by a logistic regression model with treatment and type of underlying disease as factors as covariate and by Fisher's exact tests.

All subjects in the safety analysis set were included in the analysis.

The co-primary efficacy endpoint was analysed using a restricted maximum likelihood (REML)-based mixed model for repeated measures (MMRM) approach. All subjects in the intention-to-treat (ITT) analysis set with post baseline Hb data was to be included with their observed data. The model included the fixed, categorical effects of treatment (iron isomaltoside and iron sucrose), week, treatment-by-week interaction, strata, as well as the continuous, fixed covariates of baseline Hb value and baseline Hb-by-week interaction. An unstructured (co)variance structure was used to model the within-subject errors. If, unexpectedly, this analysis failed to converge, the following structures were to be applied, in the following order; first-order ante-dependence, heterogeneous compound symmetry, compound symmetry. The Kenward-Roger approximation was to be used to estimate the denominator degrees of freedom. The primary comparisons will be the contrasts between iron isomaltoside and iron sucrose at week 8 based on the least squares means for the treatment-by-week interaction effect. The estimated mean difference based on this model was reported with two-sided symmetric 95% CI, and if the lower bound of the 95% CI is >−0.5 g/dL, the efficacy objective has been met.

### Baseline Assessment

The following was assessed by the study staff at the clinical baseline visit at site:
Inclusion and exclusion criteria reviewed to confirm that no change has occurred since screening
Pregnancy test, if applicable
Recording of relevant medical history, including history of myocardial infarction, stroke, or congestive heart failure
Recording of concomitant medication
Physical examination (not performed later than the baseline visit)
Measurement of height
Measurement of weight
Examination of vital signs
Randomisation
ECG
Assessment of fatigue by FACIT Fatigue Scale
Assessment of pharmacoeconomics by the ISDR questionnaire and health care resource use questionnaire
Safety laboratory tests
Efficacy laboratory tests
Treatment with iron isomaltoside (group A only)
Treatment with iron sucrose (group B only)
AE evaluation and recording

### Study Assessments

### Demographic and Baseline Assessments

Date of birth, gender, race, ethnicity, and smoking habits were collected. A current smoker was defined as a subject who had been smoking within the last 6 months.

### Pregnancy Test

A urine pregnancy test was performed for all women of childbearing potential. The test was handled and interpreted by the site personnel.

### Relevant Medical History

Relevant medical history was recorded. Changes in medical history were recorded at the subsequent visits during the study (worsening of symptoms or diseases were recorded as AEs). The following was collected: disease and start and stop date. Except for underlying disorder causing IDA, start dates occurring >12 months before the enrolment into the study were set as >12 months.

### Concomitant Medication

If the subject was receiving any concomitant medication it was recorded at the baseline visit. Changes in concomitant medication were recorded in the subsequent visits during the study. The following was collected: brand name, indication, route, dose, frequency, unit, and start and stop date. Start dates occurring >12 months before the enrolment into the study were set as >12 months.

### Physical Examination

A physical examination was performed based upon the Investigator's judgement and could include the following:
Head-Eyes-Ear-Nose-Throat
Cardiovascular system
Respiratory system
Nervous system
Gastrointestinal system
Musculo-skeletal system
Urogenital system
Dermatology system
Others, if required

### Height

Height was measured without shoes.

### Weight

Weight was measured.

### Vital Signs

Heart rate and blood pressure were measured at the following time points when a subject received study drug: approximately 0-10 minutes before infusion, during infusion, 5-15 minutes, and 20-40 minutes after the infusion has ended. If vital signs were measured more than once in the given time interval, the lowest measurement of diastolic blood pressure (including the attendant systolic blood pressure and heart rate) for the period was noted in the electronic Case Report Form (eCRF).

### Electrocardiogram

A standard 12 lead ECG was recorded (including date, time, and signature). At baseline and other treatment visits, two ECGs were recorded; one before administration of the study drug and one approximately 30 minutes after start of the dosing. Only one ECG was recorded at the follow-up visits.

The ECGs did not need to be evaluated by a cardiologist.

### Laboratory Assessments

It was requested that the blood samples were drawn before administering the study drug, and, if possible, that they were drawn at the same time of the day at all visits in order to reduce any diurnal fluctuation in the parameters.

Laboratory assessments were performed at a central laboratory. A Laboratory Manual was provided to each site in which all laboratory procedures were described.

### Eligibility Laboratory Assessments

The following eligibility laboratory assessments were performed:
Complete hematology set: Hb, leucocytes/White Blood Cells (WBC), erythrocytes/Red Blood Cells (RBC), haematocrit, platelets, neutrophil granulocytes, lymphocytes, monocytes, eosinophils, basophils, Mean Corpuscular Hemoglobin (MCH), Mean Corpuscular Volume (MCV), Mean Corpuscular Hemoglobin Concentration (MCHC), and reticulocyte count
Biochemistry:
   S-ferritin
   Alanine Aminotransferase (ALAT) and Aspartate Aminotransferase (ASAT)
   C-reactive Protein (CRP)
   Estimated Glomerular Filtration Rate (eGFR)

### Vitamin E

Vitamin E was measured at baseline visit as part of the demographic data.

### Safety Laboratory Assessments

The following safety laboratory assessments were analysed:
Complete haematology set: Leucocytes/WBC, erythrocytes/RBC, haematocrit, platelets, neutrophil granulocytes, lymphocytes, monocytes, eosinophils, basophils, MCH, MCV, MCHC, and reticulocyte count
Biochemistry:
   S-sodium, s-potassium, s-calcium, s-urea, s-creatinine, s-albumin
   S-bilirubin, ASAT, ALAT
   CRP

### Efficacy Laboratory Assessments

The following efficacy laboratory parameters were analysed:
Hb: Hemoglobin was analyzed by the Coulter LH 750 System. Lytic reagents were used for the complete blood count parameters to prepare the blood so that the system could measure the amount of hemoglobin. The lytic reagent rapidly and simultaneously destroyed the erythrocytes and converted the substantial proportion of the hemoglobin to a stable pigment. The absorbance of the pigment was directly proportional to the hemoglobin concentration in the sample. The accuracy of this method equaled that of the hemoglobin cyanide method.
S-ferritin: The Access ferritin assay was a two-site immunoenzymatic (“sandwich”) assay. A sample was added to a reaction vessel with goat anti-ferritin-alkaline phosphatase conjugate, and paramagnetic particles coated with goat anti-mouse: mouse anti-ferritin complexes. Serum or plasma (heparin) ferritin binds to the immobilized monoclonal anti-ferritin on the solid phase, while the goat anti-ferritin enzyme conjugate reacts with different antigenic sites on the ferritin molecules. Separation in a magnetic field and washing removed materials not bound to the solid phase. A chemiluminescent substrate, Lumi-Phos* 530, was added to the reaction vessel and light generated by the reaction was measured with a luminometer.
TSAT (s-iron and transferrin will be collected to calculate the TSAT; TSAT=(iron μg/dL/transferrin mg/dL)×70.9).
S-iron: Serum iron (s-iron) was measured by a calorimetric assay using Roche automated clinical chemistry analyzer based on a immunoturbidimetric assay.

Adverse Events (AE)

AEs data were collected and evaluated for relatedness to study drug, seriousness, severity, and expectedness.

As summarized in the table below, a total of N=260 patients (N=168 in the IIM group; N=92 in the IS group) had been diagnosed with CHF according to their medical history.

Patients diagnosed with CV risk according to their medical history were N=518 in total (N=345 in the IIM group; N=173 in the IS group). A total of N=226 patients thereof (N=144 in the IIM group; N=82 in the IS group) has been diagnosed with CV risk and CHF according to their medical history. Patients diagnosed with CV risk without CHF have been N=292 in total (N=144 in the IIM group; N=82 in the IS group). A total of N=2748 have been patients without CHF (N=144 in the IIM group; N=82 in the IS group).

| | CKD-04 | | | | IDA-03 | | | | CKD-04/IDA-03 | | | |
|---|---|---|---|---|---|---|---|---|---|---|---|---|
| | Iron | | Iron | | Iron | | Iron | | Iron | | Iron | |
| | isomaltoside | | sucrose | | isomaltoside | | sucrose | | isomaltoside | | sucrose | |
| Population | N | % | N | % | N | % | N | % | N | % | N | % |
| All patients | [symbol] | 100.0 | [symbol] | 100.0 | 989 | 100.0 | [symbol] | 100.0 | [symbol] | 100.0 | 100.0 | 100.0 |
| All patients with CHF | [symbol] | [symbol] | [symbol] | [symbol] | [symbol] | [symbol] | [symbol] | [symbol] | [symbol] | [symbol] | [symbol] | [symbol] |
| All patients without CHF | [symbol] | [symbol] | [symbol] | [symbol] | [symbol] | [symbol] | [symbol] | [symbol] | [symbol] | [symbol] | [symbol] | [symbol] |
| Patients with CV risk | [symbol] | [symbol] | [symbol] | [symbol] | [symbol] | [symbol] | [symbol] | [symbol] | [symbol] | [symbol] | 173 | [symbol] |

CV risk: (Part of) Stratfication factor in the trielo History of myocardial infarction, stroke, or congestive heart failure (CHF)
CHF: Patients with congestive heart failure in medical history [symbol]
indicates data missing or illegible when filed

### Results

Key findings include a clear impact of iron isomaltoside 1000 on treatment-emergent congestive heart failure adverse events in patients having a risk of cardiovascular adverse events.

In total 1525 patients were enrolled in the CKD-04 study and treated with either isomaltoside 1000 or iron sucrose. 4.1% of the 1019 patients treated with iron isomaltoside 1000 in the CKD-04 study had composite cardiovascular events, while the incidence was 6.9% in the iron sucrose treatment group (506 patients).

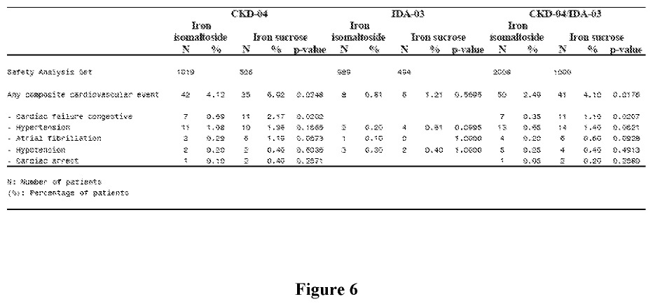

In total 1525 patients were enrolled in the IDA-03 trail and treated with either isomaltoside 1000 or iron sucrose 0.8% of the 989 patients treated with iron isomaltoside 1000 and 1.2% of the 494 patients treated with iron experienced composite cardiovascular events. In the combined CKD-04/IDA-03 study, composite cardiovascular events were observed in 2.5% of the 2008 patients treated with iron isomaltoside 1000 versus 4.1% in iron sucrose (p=0.0176), resulting in a decrease of composite cardiovascular events of 60% when patients are treated with iron isomaltoside 1000. In each study, the rates of congestive heart failure, hypertension, atrial fibrillation, hypotension, and cardiac arrest were lower in the iron isomaltoside 1000 treatment group compared to the iron sucrose treatment group. See FIG. 6.

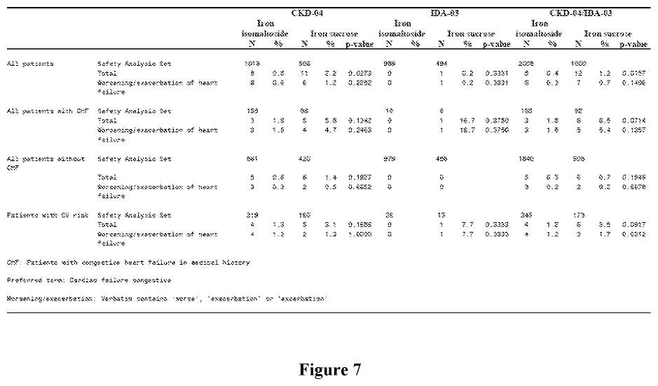

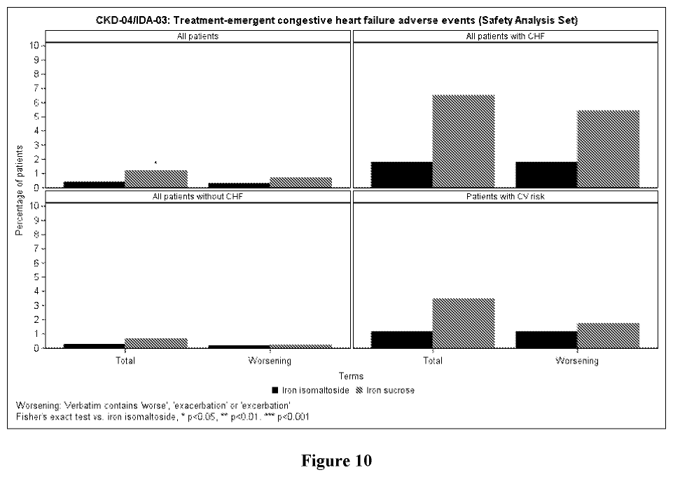

The total patient population has been separated into different subgroups of patients: all patients, all patients with or without CHF, patients with CV risk and patients with CV risk with or without CHF. In each patient group, the incidence of treatment-emergent congestive heart failure adverse events was similar and in most cases significant lower using iron isomaltoside 1000 compared to treatment with iron sucrose. See FIG. 7. For instance, in the combined study CKD-04/IDA-03, 6.5% of all patients with CHF had treatment-emergent congestive heart failure adverse events when treated with iron sucrose, whereas treatment with iron isomaltoside 1000 resulted in only 1.8% of said patient group experiencing such adverse events. Worsening/exacerbation of heart failure occurred in 5.4% of the cases using iron sucrose in said patient group compared to only 1.8% when treated with iron isomaltoside 1000. Similar significant reductions in treatment-emergent congestive heart failure adverse events were observed. In patients with CV risk, the incidence for congestive heart failure was 1.2% in the IIM-treatment group and 3.5% in the IS-treatment group, with a worsening of congestive heart failure occurring in 1.2% of the IIM-treatment group and 1.7% of the IS-treatment group. In patients with CV risk and CHF, the incidence for congestive heart failure was 1.4% in the IIM-treatment group and 4.9% in the IS-treatment group, with a worsening of congestive heart failure occurring in 1.4% of the IIM-treatment group and 3.7% of the IS-treatment group. The significance of the results of said study is further illustrated in FIG. 10 which exemplarily presents the data for the combined CKD-04/IDA-03 study in form of a bar chart.

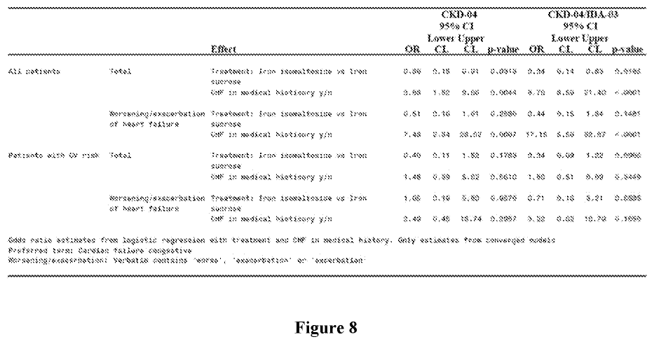

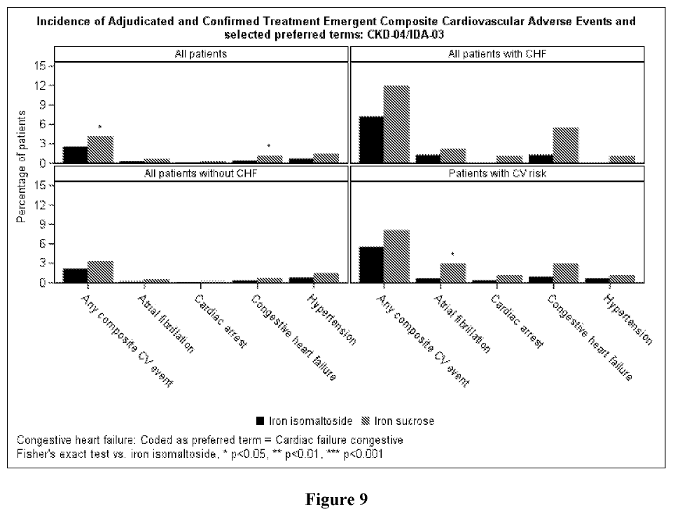

The odds ratios for the treatment with iron isomaltoside 1000 versus iron sucrose are shown in FIG. 8. Odds ratios <1 indicate a probability for a treatment-emergent congestive heart failure adverse event that is lower for the treatment with iron isomaltoside 1000 than for the treatment with iron sucrose. See FIG. 9.

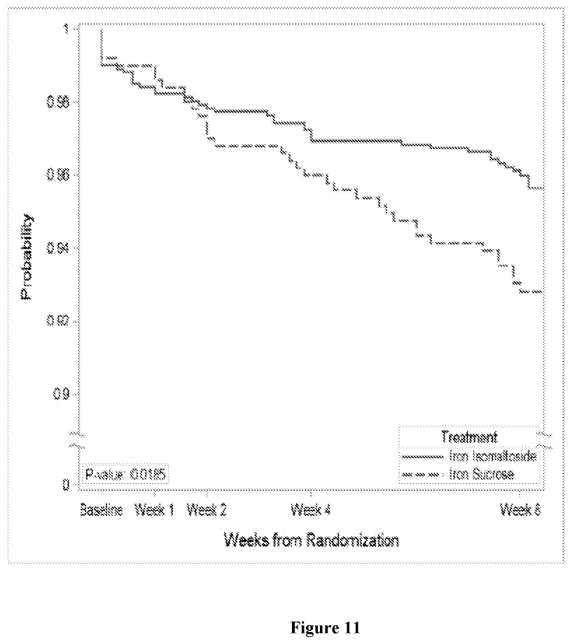

The probability of a patient (CKD-04) not experiencing an adjudicated composite adverse cardiovascular event is significantly higher after 8 weeks of treatment with iron isomaltoside 1000 than treatment with iron sucrose. See FIG. 11.

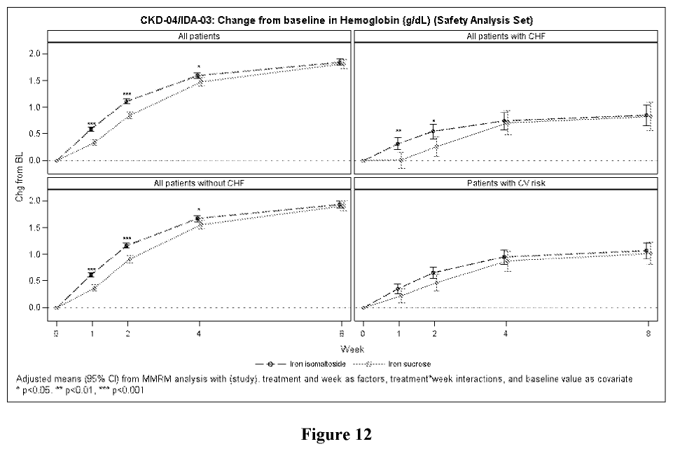

In the combined CKD-04/IDA-03 study (all patients), iron isomaltoside 1000 led to a higher increase in Hb from baseline in weeks 1 and 2 (p<0.001) and non-inferiority was demonstrated for change in Hb from baseline in weeks 4 and 8 (primary efficacy endpoint). See FIG. 12.

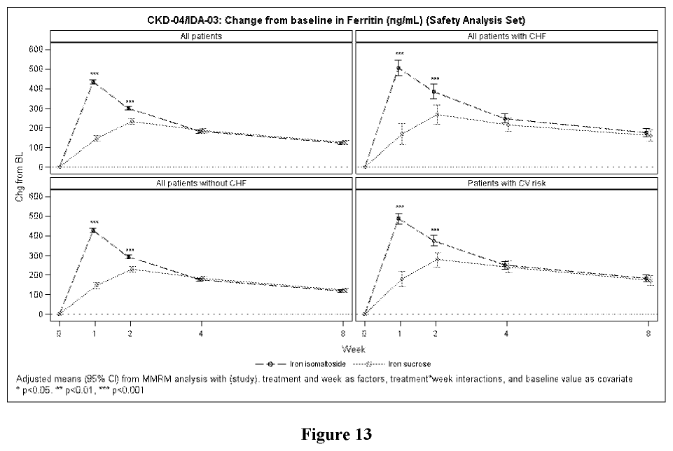

FIG. 13 shows the change from baseline in ferritin (ng/mL) by patients with CHF in medical history for the combined studies CKD-04/IDA-03 (iron isomaltoside 1000: dashed line, iron sucrose: full line).

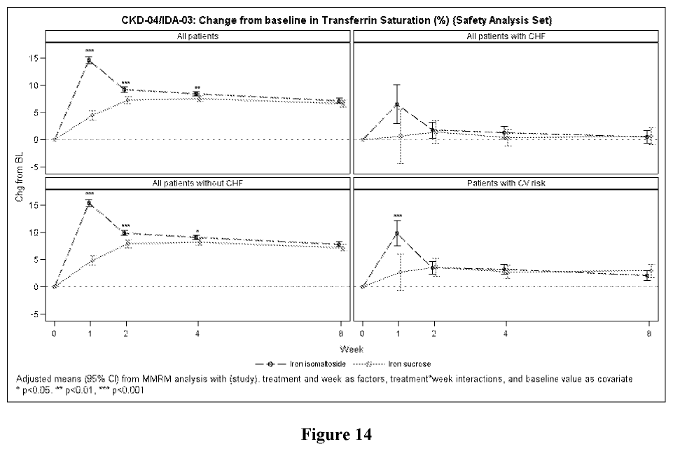

FIG. 14 shows the change from baseline in TSAT (%) by patients with CHF in medical history for the combined studies CKD-04/IDA-03 (iron isomaltoside 1000: dashed line, iron sucrose: full line).

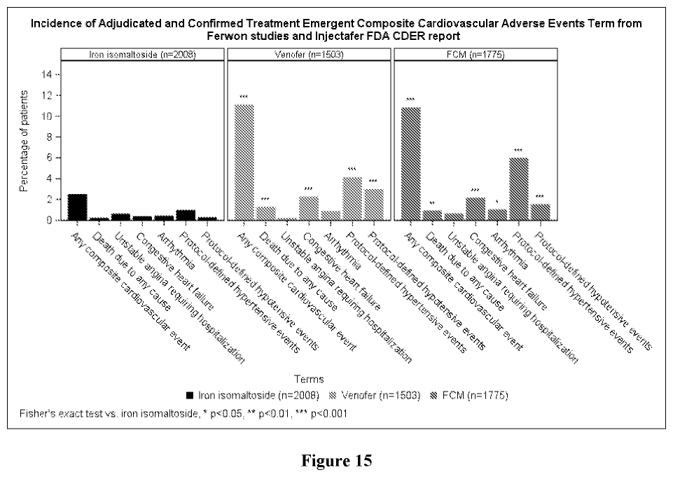

FIG. 15 shows the percentage of patients who experienced treatment-emergent composite CV adverse events after treatment with IIM 1000, Venofer and FCM (combined studies CKD-04/IDA-03 for TIM 1000 (Ferwon studies); studies using Venofer and FCM based on Injectafer FDA CDER report).

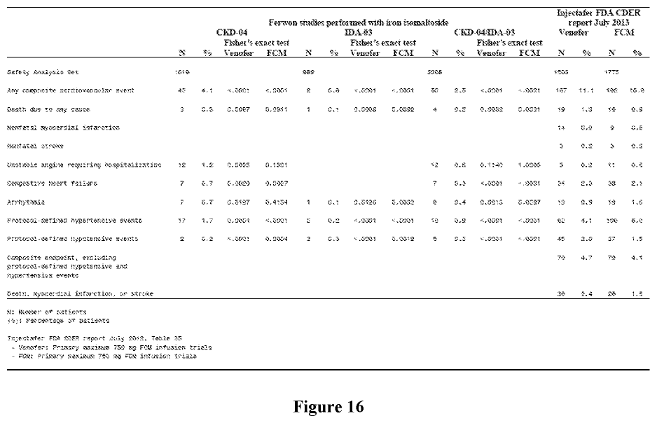

FIG. 16 is a table summarizing the analysis of adverse events for study CKD-04, study IDA-03, and the combined studies CKD-04/IDA-03 using IIM 1000 (Ferwon studies) compared to studies using Venofer and FCM (Injectafer FDA CDER report).

In summary, the studies demonstrate that the incidence of cardiovascular adverse events is lower for treatment with iron isomaltoside 1000 compared to iron sucrose in CKD-04 and in the pooled analysis across CKD-04 and IDA-03 on the composite cardiovascular end point as well as congestive heart failure in particular. Further, CV adverse event incidence rates and numerical differences to iron sucrose are in general higher in CHF patient subgroups (CHF in medical history/CV risk factor and CHF). Compared to Venofer and FCM (CDER report), the incidence on composite endpoint (and most sub-items including death from any cause) are lower for treatment with iron isomaltoside 1000.

Treatment with iron isomaltoside leads to a higher increase in Hb from baseline to week 1 and 2 and to a similar response at week 8 in patients with congestive heart failure in medical history.

### Example 2

A study was carried out to establish the effect of iron on P-wave dispersion.

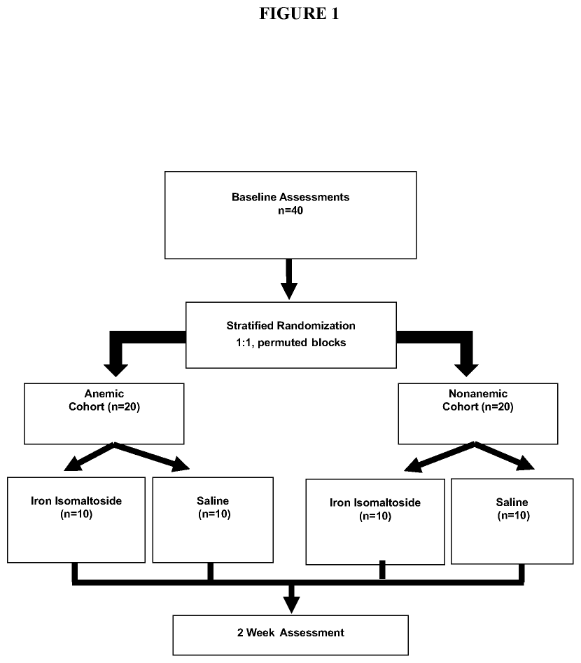

This study was a post-hoc analysis of a randomized, double-blind, placebo-controlled trial that was approved by the South-Central Berkshire ethics committee, the UK Medicines and Healthcare products Regulatory Agency, and the King's College Hospital independent research governance board. King's College London University and King's College Hospital NHS Foundation Trust were co-sponsors. The King's Health Partners clinical trial office monitored the trial and ensured compliance with the International Conference on Harmonization guidelines for Good Clinical Practice and the Declaration of Helsinki. Written informed consent was obtained from all patients. FIG. 1 represents a flow chart of the trial. A total of 25 chronic heart failure patients in normal sinus rhythm were recruited into this trial with 15 randomized to intravenous saline placebo and 10 randomized to intravenous iron isomaltoside.

Patients with stable, symptomatic, chronic heart failure and a left ventricular ejection fraction <45% were recruited from dedicated heart failure clinics at King's College Hospital, London. Main exclusion criteria were a history of acquired iron overload, known haemochromatosis or first degree relatives with haemochromatosis; an allergic disorder (e.g., asthma, eczema, and anaphylactic reactions); prior hypersensitivity to IV iron drugs or their excipients; active infection, bleeding, malignancy, haemolytic anemia, rheumatoid arthritis, and myelodysplasia; and HIV/AIDS.

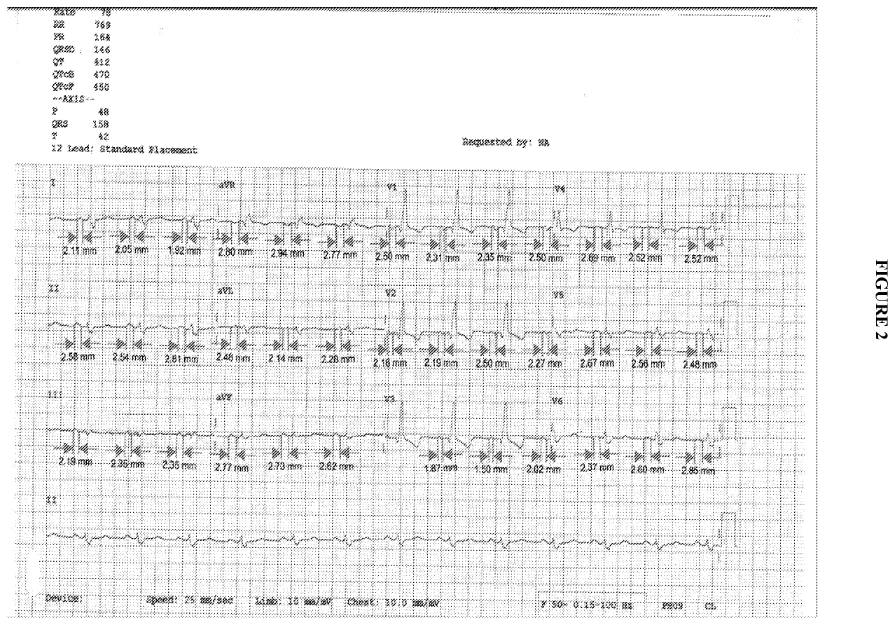

After inclusion into the trial, patients had endpoints assessed which included a 12-lead surface electrocardiogram to measure P-wave dispersion (difference between maximum and minimum P-wave duration across all leads), heart rate (HR), PR interval, QRS duration, QT interval and corrected QT interval (QTc). FIG. 2 is an example of a surface electrocardiogram with P-wave durations measured. An echocardiogram was also done to measure LVEF, left ventricular end-diastolic volume (LVED), left ventricular end-systolic volume (LVES), left atrial (LA) volumes and right atrial (RA) volumes with and without indexing for body surface area. After baseline assessments, patients were then infused a total repletion dose of iron isomaltoside or an equivalent volume of normal saline placebo. Endpoints were then reassessed 2 weeks later. A Student's t-test or a Fisher's Exact test were statistically used to assess the baseline characteristics between the groups. An ANCOVA was used to statistically compare the week 2 endpoint results between the groups with adjustment for each variables baseline value.

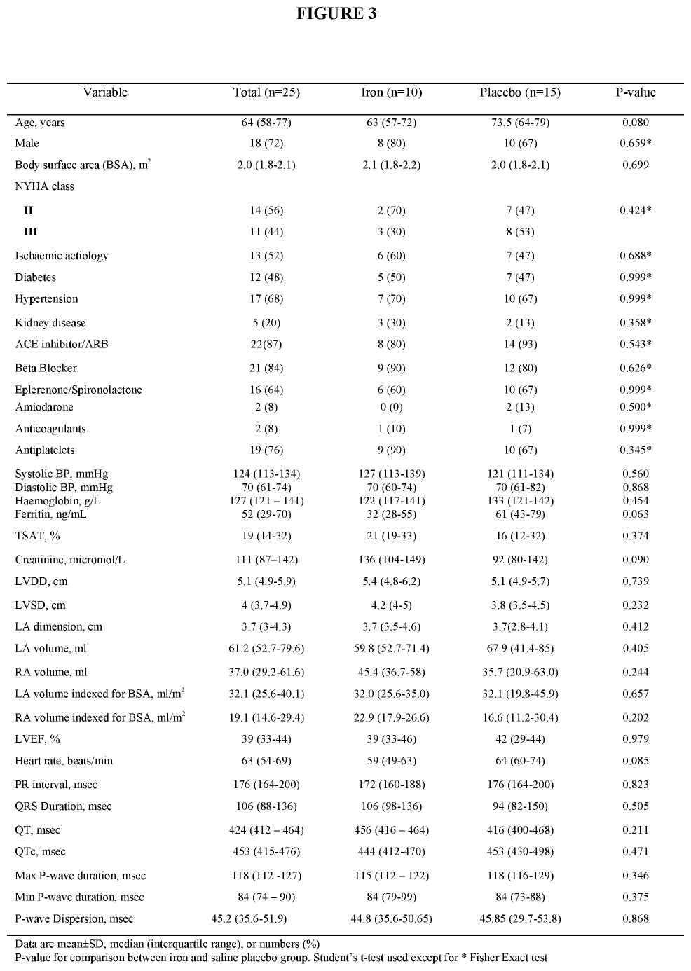

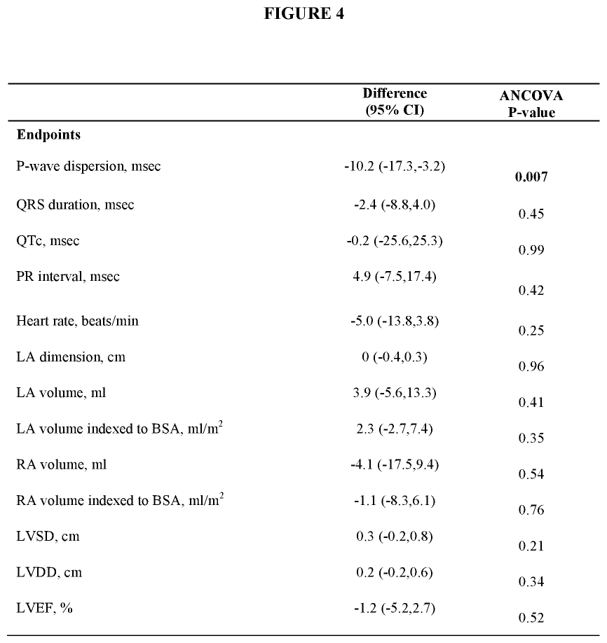

The baseline characteristics of the 2 groups are shown in FIG. 3. The groups were well matched for these baseline characteristics. FIG. 4 shows the effect of iron on each endpoint at 2 weeks using an ANCOVA statistical analysis. At 2 weeks, the use of iron had no significant effect on any of the endpoints except for P-wave dispersion. Iron treatment significantly reduced P-wave dispersion by 10 msec (adjusted difference between the groups −10 msec, 95% confidence interval −17 msec & −3 msec, P=0.007) at 2 weeks. The use of iron was still associated with a significant reduction in P-wave dispersion after adjusting for the size of the atria using indexed LA and RA volumes (−11 msec, 95% confidence interval −18 msec & −4 msec, P=0.005). Thus, this data shows that iron administration was associated with a reduction in P-wave dispersion, and a reduction in P-wave dispersion is needed for the prevention and treatment of AF.

### EQUIVALENTS

Those skilled in the art will recognize, or be able to ascertain using no more than routine experimentation, many equivalents of the specific embodiments disclosed herein. Such equivalents are intended to be encompassed by the following claims.

### NON-PATENT PUBLICATIONS

Anker S D, Comin Colet J, Filippatos G, Willenheimer R, Dickstein K, Drexler H, Luscher T F, Bart B, Banasiak W, Niegowska J, Kirwan B A, Mori C, von Eisenhart Rothe B, Pocock S J, Poole-Wilson P A, Ponikowski P. Ferric carboxymaltose in patients with heart failure and iron deficiency. N Engl J Med 2009; 361:2436-2448.
Ponikowski P, van Veldhuisen D J, Comin-Colet J, Ertl G, Komajda M, Mareev V, McDonagh T, Parkhomenko A, Tavazzi L, Levesque V, Mori C, Roubert B, Filippatos G, Ruschitzka F, Anker S D. Beneficial effects of long-term intravenous iron therapy with ferric carboxymaltose in patients with symptomatic heart failure and iron deficiency. Eur Heart J 2015; 36:657-668.
Jankowska E A, Kasztura M, Sokolski M, Bronisz M, Nawrocka S, Oles'kowska-Florek W, Zymlin'ski R, Biegus J, Siwolowski P, Banasiak W, Anker S D, Filippatos G, Cleland J G F, Ponikowski P. Iron deficiency defined as depleted iron stores accompanied by unmet cellular iron requirements identifies patients at the highest risk of death after an episode of acute heart failure. Eur Heart J 2014; 35:2468-2476.
Ponikowski P, Voors A A, Anker S D, Bueno H, Cleland J G, Coats A J, Falk V, Gonzalez-Juanatey J R, Harjola V P, Jankowska E A, Jessup M, Linde C, Nihoyannopoulos P, Parissis J T, Pieske B, Riley J P, Rosano G M, Ruilope L M, Ruschitzka F, Rutten F H, van der Meer P; Authors/Task Force Members; Document Reviewers. 2016 ESC Guidelines for the diagnosis and treatment of acute and chronic heart failure: The Task Force for the diagnosis and treatment of acute and chronic heart failure of the European Society of Cardiology (ESC). Eur J Heart Fail. 2016 August; 18(8):891-975. doi: 10.1002/ejhf.592. Epub 2016 May 20.
Yancy C W, Jessup M, Bozkurt B, Butler J, Casey D E Jr, Colvin M M, Drazner M H, Filippatos G S, Fonarow G C, Givertz M M, Hollenberg S M, Lindenfeld J, Masoudi F A, McBride P E, Peterson P N, Stevenson L W, Westlake C. 2017 ACC/AHA/HFSA Focused Update of the 2013 ACCF/AHA Guideline for the Management of Heart Failure: A Report of the American College of Cardiology/American Heart Association Task Force on Clinical Practice Guidelines and the Heart Failure Society of America. J Am Coll Cardiol. 2017 Aug. 8; 70(6):776-803. doi: 10.1016/j.jacc.2017.04.025. Epub 2017 Apr. 28.
Myles Wolf, M D, Janet Rubin, M D, Maureen Achebe, M D, Michael John Econs, M D, Munro Peacock, M D, Erik Allen Imel, M D, Lars L. Thomsen, M D, Thomas O. Carpenter, M D, Thomas Joseph Weber, M D, Heinz Zoller, M D. Effects of Iron Isomaltoside versus Ferric Carboxymaltose on Hormonal Control of Phosphate Homeostasis: The PHOSPHARE IDA04/05 Randomized Controlled Trials. ENDO 2019, Mar. 23-26, 2019, New Orleans, Session OR13-OR13. Rare Bone Diseases and Mineral Metabolism, abstract OR13-3.
HILDEBRANDT, P. R., BRUUN, N. E., NIELSEN, 0. W., PANTEV, E., SHIVA, F., VIDEBÆK, L., WIKSTRÖM, G. and THOMSEN, L. L. (2010), Effects of administration of iron isomaltoside 1000 in patients with chronic heart failure. A pilot study. Transfusion Alternatives in Transfusion Medicine, 11: 131-137. doi:10.1111/j.1778-428X.2010.01145.x
Charles-Edwards G, Amaral N3, Sleigh A, Ayis S, Catibog N3, McDonagh T3, Monaghan M, Amin-Youssef G, Kemp G J, Shah A M, Okonko D O. Effect of Iron Isomaltoside on Skeletal Muscle Energetics in Patients With Chronic Heart Failure and Iron Deficiency. Circulation. 2019 May 21; 139(21):2386-2398. doi: 10.1161/CIRCULATIONAHA.118.038516.
Hannah Jaumdally, Mohamad F. Barakat, Geoffrey Charles-Edwards, GeorgeAmin-Youssef, Ajay M. Shah, Paul Scott, Darlington O. Okonko. IRON ISOMALTOSIDE DIMINISHES ATRIAL ELECTRICAL INHOMOGENEITY IN CHRONIC HEART FAILURE WITHOUT ALTERING ATRIAL SIZE: A FERRIC-HF II SUBSTUDY. Journal of the American College of Cardiology March 2019, 73 (9 Supplement 1) 833; DOI: 10.1016/S0735-1097(19)31440-8

1. A method of treating iron deficiency in a subject, wherein the treatment of iron deficiency reduces the incidence of, or risk for, a cardiovascular adverse event in the subject, which method comprises administering an effective amount of iron isomaltoside,
wherein the subject is:
(A) a subject being at risk of a cardiovascular adverse event;
(B) a subject having a history of congestive heart failure (CHF); or
(C) a subject having a history of congestive heart failure (CHF) and being at risk of a cardiovascular adverse event.

2. The method of claim 1, wherein the cardiovascular adverse event, the incidence of, or risk for, which reduced is:
(a) selected from the group consisting of congestive heart failure, myocardial infarction, unstable angina, arrhythmia, hypertension, hypotension, stroke, and death;
(b) congestive heart failure;
(c) atrial fibrillation;
(d) hypertension; or
(e) cardiac arrest.

3. The method of claim 1, wherein:
(a) the subject is a subject being at risk of a cardiovascular adverse event and the cardiovascular adverse event, the incidence of, or risk for, which is reduced, is congestive heart failure;
(b) the subject is a subject being at risk of a cardiovascular adverse event and the cardiovascular adverse event, the incidence of, or risk for, which is reduced, is atrial fibrillation;
(c) the subject is a subject being at risk of a cardiovascular adverse event and the cardiovascular adverse event, the incidence of, or risk for, which is reduced, is cardiac arrest;
(d) the subject is a subject having a history of congestive heart failure and the cardiovascular adverse event, the incidence of, or risk for, which is reduced, is cardiac arrest or congestive heart failure or both;
(e) the subject is a subject having a history of congestive heart failure (CHF) and the cardiovascular adverse event, the incidence of, or risk for, which is reduced, is atrial fibrillation; or
(f) the subject is a subject having a history of congestive heart failure (CHF) and the cardiovascular adverse event, the incidence of, or risk for, which is reduced, is cardiac arrest.

4-8. (canceled)

9. The method of claim 1, wherein the subject is a subject being at risk of a cardiovascular adverse event and/or having a history of congestive heart failure, wherein the subject has chronic kidney disease (CKD); or wherein the subject is a subject being at risk of a cardiovascular adverse event and/or having congestive heart failure, wherein the subject does not have chronic kidney disease (CKD).

10. (canceled)

11. The method of claim 9, wherein the chronic kidney disease (CKD) is non-dialysis dependent chronic kidney disease (NDD-CKD).

12. The method of claim 1, wherein:
(a) the subject having a history of congestive heart failure has HFrEF;
(b) the subject having a history of congestive heart failure has congestive heart failure in NYHA class II-IV; and/or
(c) the cardiovascular adverse event, the incidence of, or risk for, which is reduced, is cardiovascular death and/or hospitalization due to worsening congestive heart failure.

13-14. (canceled)

15. The method of claim 1, wherein the iron deficiency is defined as TSAT <20% and/or ferritin <100 μg/L.

16. The method of claim 1, wherein the iron isomaltoside is ferric derisomaltose.

17. The method of claim 1, wherein the subject is also being treated with another agent used to treat or prevent cardiovascular adverse events including but not limited to congestive heart failure (CHF), cardiac arrest, congestive heart failure (CHF), myocardial infarction, unstable angina, arrhythmia, hypertension, hypotension, stroke, and atrial fibrillation.

18. A method of reducing P-wave dispersion/duration for the prevention or treatment of atrial fibrillation (AF) or disorders that predispose to AF in an animal suffering from such a condition which comprises administering to such an animal a therapeutically effective amount of an iron agent.

19. The method of claim 18, wherein said disorders that predispose to AF are selected from heart valve disease, hypertension, heart failure, coronary artery disease, obesity, and diabetes mellitus.

20. The method of claim 18, wherein said animal is a mammal, or wherein said animal is human.

21. (canceled)

22. The method of claim 18, wherein said AF is paroxysmal, persistent, long-standing, or chronic.

23. The method of claim 18, wherein said iron agent is administered via the oral, intramuscular, or intravenous route.

24. The method of claim 18, wherein said animal is iron deficient.

25. The method of claim 18, wherein said animal is not iron deficient.

26. The method of claim 18, which reduces AF symptoms such as palpitations or breathlessness, exercise intolerance, hospitalization, heart failure, stroke and death.

27. The method of claim 18, wherein said iron agent is an oral tablet, intramuscular injection, or intravenous injection or infusion.

28. The method of claim 27, wherein said oral tablet comprises ferrous sulphate, ferrous fumarate, ferrous gluconate, iron polysaccharide, or iron polymaltose; or wherein said intravenous injection or infusion comprises high molecular weight iron dextran, low molecular weight iron dextran, iron sucrose, ferrous gluconate, ferric carboxymaltose, or iron isomaltoside.

29. (canceled)

30. The method of claim 18, wherein said animal has myocardial iron deficiency; and/or wherein said myocardial iron deficiency responds to supplementation despite having normal blood tests for iron deficiency.

31. (canceled)
